(19) United States
(12) Patent Application Publication
(10) Pub. No.: US 2020/0347064 A1
(43) Pub. Date: Nov. 5, 2020

(54) SYNTHESIS OF A BRUTON'S TYROSINE KINASE INHIBITOR

(71) Applicants: Pharmacyclics LLC, Sunnyvale, CA (US); Janssen Pharmaceutica NV, Beerse (BE)

(72) Inventors: Cyril Ben Haim, Beerse (BE); Wei Chen, Fremont, CA (US); Erick Goldman, Concord, CA (US); Andras Horvath, Beerse (BE); Philip Pye, Beerse (BE); Mark S. Smyth, Foster City, CA (US); Erik J. Verner, San Mateo, CA (US)

(21) Appl. No.: 16/703,344

(22) Filed: Dec. 4, 2019

Related U.S. Application Data
(63) Continuation of application No. 16/224,425, filed on Dec. 18, 2018, now abandoned.
Application No. 16/224,425 is a continuation of application No. 15/542,848, filed on Mar. 2, 2018, now abandoned.
(60) Provisional application No. 62/103,507, filed on Jan. 14, 2015.

(51) Int. Cl. C07D 487/04

(52) U.S. Cl. CPC C07D 487/04

(57) ABSTRACT

Described herein is the synthesis of Bruton's tyrosine kinase (Btk) inhibitor 1-((R)-3-(4-amino-3-(4-phenoxyphenyl)-1H-pyrazolo[3,4-d]pyrimidin-1-yl)piperidin-1-yl)prop-2-en-1-one.

### CROSS-REFERENCE TO RELATED APPLICATION

This application claims the benefit of U.S. Provisional Application No. 62/103,507, filed Jan. 14, 2015, which is incorporated herein by reference in its entirety.

### BACKGROUND OF THE INVENTION

Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses.

Btk is a key regulator of B-cell development, activation, signaling, and survival. In addition, Btk plays a role in a number of other hematopoietic cell signaling pathways, e.g., Toll like receptor (TLR) and cytokine receptor-mediated TNF-α production in macrophages, IgE receptor (Fc epsilon RI) signaling in mast cells, inhibition of Fas/APO-1 apoptotic signaling in B-lineage lymphoid cells, and collagen-stimulated platelet aggregation.

1-((R)-3-(4-amino-3-(4-phenoxyphenyl)-1H-pyrazolo[3,4-d]pyrimidin-1-yl)piperidin-1-yl)prop-2-en-1-one (ibrutinib) is a Bruton's tyrosine kinase (Btk) inhibitor. 1-((R)-3-(4-amino-3-(4-phenoxyphenyl)-1H-pyrazolo[3,4-d]pyrimidin-1-yl)piperidin-1-yl)prop-2-en-1-one is also known by its IUPAC name as 1-{(3R)-3-[4-amino-3-(4-phenoxyphenyl)-1H-pyrazolo[3,4-d]pyrimidin-1-yl]piperidin-1-yl}prop-2-en-1-one or 2-Propen-1-one, 1-[(3R)-3-[4-amino-3-(4-phenoxyphenyl)-1H-pyrazolo[3,4-d]pyrimidin-1-yl]-1-piperidinyl-, and has been given the USAN name, ibrutinib. The various names given for ibrutinib are used interchangeably herein.

### SUMMARY OF THE INVENTION

Described herein is the synthesis of the Btk inhibitor 1-((R)-3-(4-amino-3-(4-phenoxyphenyl)-1H-pyrazolo[3,4-d]pyrimidin-1-yl)piperidin-1-yl)prop-2-en-1-one (ibrutinib) (Formula (I)):

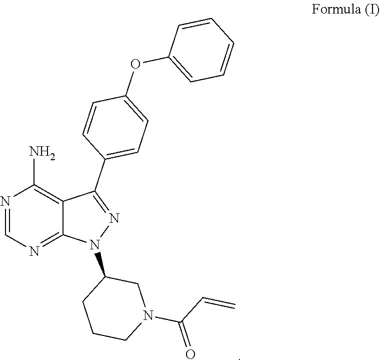 [SMILES: C=CC(=O)N1CCC[C@@H](n2nc(-c3ccc(Oc4ccccc4)cc3)c3c(N)ncnc32)C1]

In one aspect, provided is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), which process comprises reacting a compound of Formula (II) with the compound of Formula (III) wherein X is a halogen, boronic acid or boronic ester such as —B(OR⁵)₂, wherein each R⁵ is independently H or alkyl, or two R⁵ together with the B and O atoms to which they are attached form a cyclical structure:

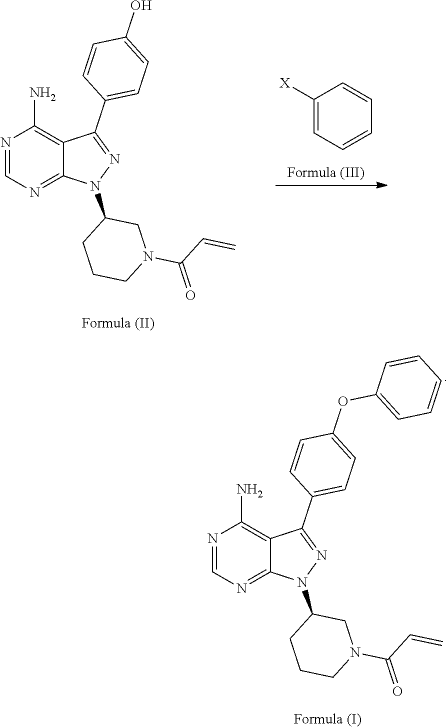

In a further embodiment described herein, the reacting the compound of Formula (II) with a compound of Formula (III) is in the presence of a catalyst, such as a copper salt. Other catalytic species which may be utilized include, but are not limited to, catalysts comprising copper, nickel, titanium or palladium, such as salts, oxides, and complexes of copper, nickel, titanium or palladium.

In some embodiments, two R⁵ together form an alkylene.

In one aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting the compound of Formula (II) with phenylboronic acid:

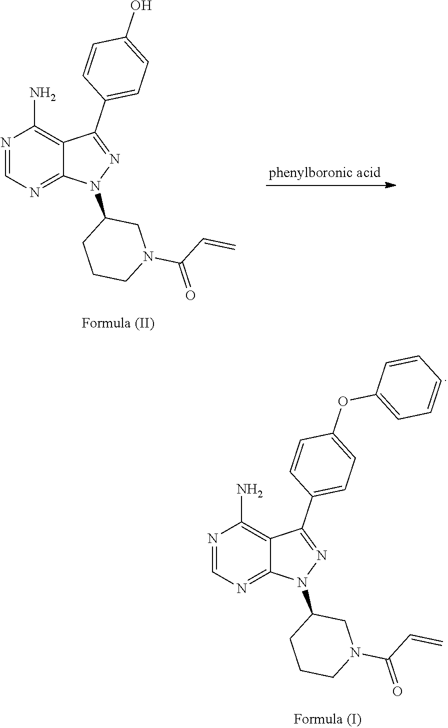 [SMILES: C=CC(=O)N1CCC[C@@H](n2nc(-c3ccc(O)cc3)c3c(N)ncnc32)C1 ; C=CC(=O)N1CCC[C@@H](n2nc(-c3ccc(Oc4ccccc4)cc3)c3c(N)ncnc32)C1]

In a further embodiment described herein, the process comprises reacting the compound of Formula (II) with phenylboronic acid in the presence of a catalyst, such as a copper salt (e.g., copper (II) acetate) and a base. In some embodiments, the base is an inorganic base, such as MOH, M₂CO₃ (wherein M is selected from lithium, sodium, potassium, and cesium), CaCO₃, di- and tri-basic phosphates (e.g. M₃PO₄, M₂HPO₄) or bicarbonates (MHCO₃). In some embodiments, the base is an organic base, such as tri-substituted amine, pyridine or 4-dimethylaminopyridine. In some embodiments, the base is NR₁R₂R₃ wherein R₁, R₂, and R₃ are each independently C₁-C₆alkyl, such as triethylamine.

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (II) with the compound of Formula (III) wherein X is a halogen:

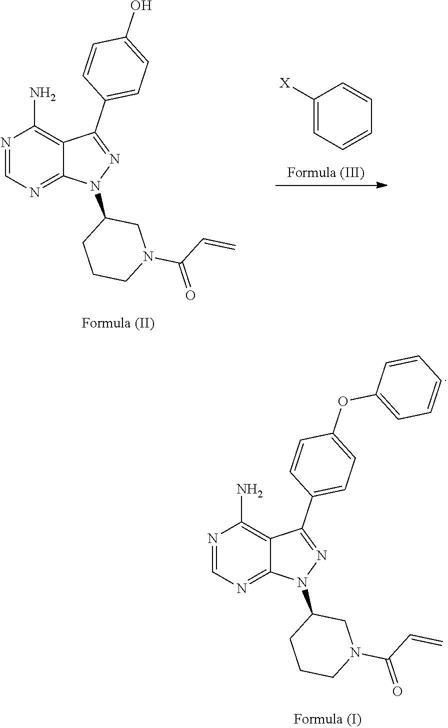

In a further embodiment described herein, the process comprises reacting the compound of Formula (II) with a compound of Formula (III) wherein X is a halogen, in the presence of a catalyst, such as copper salts (e.g., copper (II) acetate) and a base. In some embodiments, the base is an inorganic base such as MOH, M₂CO₃ (wherein M is selected from lithium, sodium, potassium, and cesium), CaCO₃, di- and tri-basic phosphates (e.g. M₃PO₄, M₂HPO₄) or bicarbonates (MHCO₃). In some embodiments, the base is an organic base, such as tri-substituted amine, pyridine or 4-dimethylaminopyridine. In some embodiments, the base is NR₁R₂R₃ wherein R₁, R₂, and R₃ are each independently C₁-C₆alkyl, such as triethylamine Other catalytic species which may be utilized include, but are not limited to, salts, oxides, and complexes of copper, nickel, titanium or palladium.

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (IV), wherein X is a halogen, with phenol:

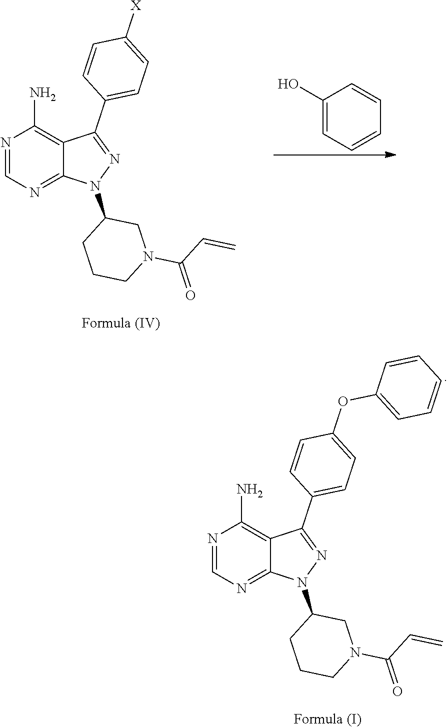

In a further embodiment described herein, the process comprises reacting the compound of Formula (IV), wherein X is a halogen, with phenol in the presence of a catalyst, such as copper salts (e.g., copper (II) acetate) and a base. In some embodiments, the base is an inorganic base such as MOH, M₂CO₃ (wherein M is selected from lithium, sodium, potassium, and cesium), CaCO₃, di- and tri-basic phosphates (e.g. M₃PO₄, M₂HPO₄) or bicarbonates (MHCO₃). In some embodiments, the base is an organic base, such as tri-substituted amine, pyridine or 4-dimethylaminopyridine. In some embodiments, the base is NR₁R₂R₃ wherein R₁, R₂, and R₃ are each independently C₁-C₆alkyl, such as triethylamine. Other catalytic species which may be utilized include, but are not limited to, salts, oxides, and complexes of copper, nickel, titanium or palladium.

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (V), wherein L is a leaving group, with ammonia:

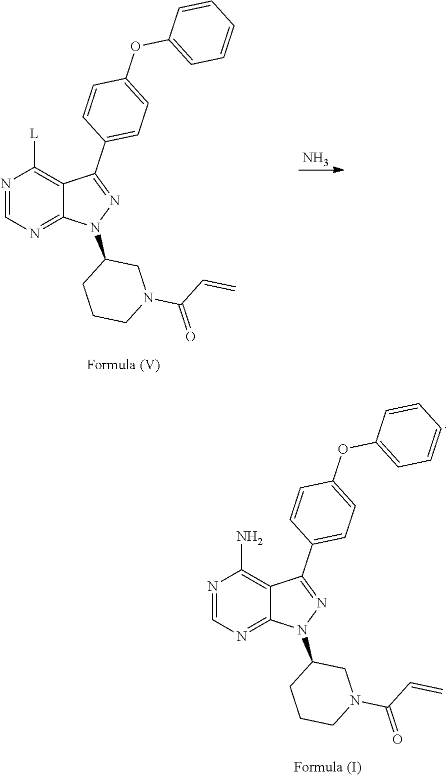

In some embodiments, L is halogen, hydroxy, alkoxy, —P(═O)R⁶₂ (wherein R⁶ is independently OH, OR⁷ (R⁷ is alkyl) or halo (e.g. Cl)), methanesulfonate (mesylate) or trifluoromethanesulfonate. In a further embodiment described herein, the process comprises reacting a compound of Formula (V), wherein L is halogen, hydroxy, alkoxy, or trifluoromethanesulfonate, with ammonia. In another embodiment, L is dichlorophosphate (—P(═O)Cl₂).

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reducing the compound of Formula (VI):

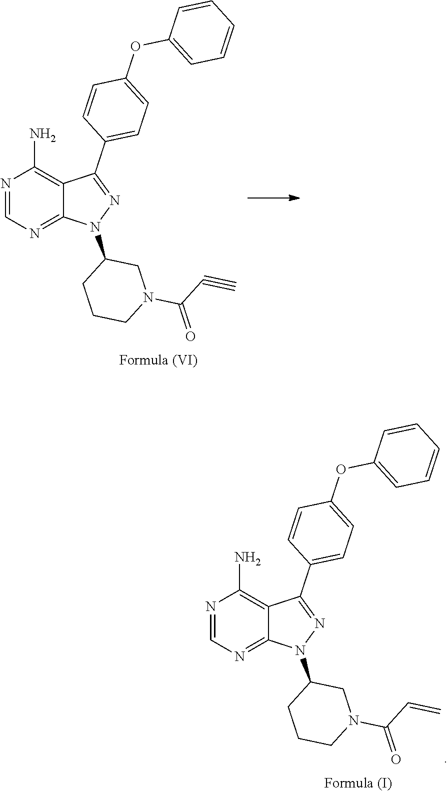 [SMILES: C#CC(=O)N1CCC[C@@H](n2nc(-c3ccc(Oc4ccccc4)cc3)c3c(N)ncnc32)C1 ; C=CC(=O)N1CCC[C@@H](n2nc(-c3ccc(Oc4ccccc4)cc3)c3c(N)ncnc32)C1]

In a further embodiment described herein, the process comprises reducing the compound of Formula (VI) by catalytic hydrogenation.

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reducing a compound of Formula (VII) wherein Z is halogen or trifluoromethanesulfonate:

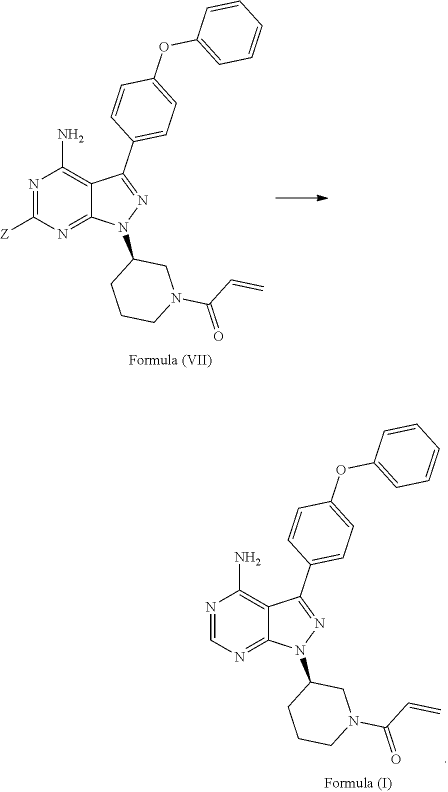

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reducing a compound of Formula (VIII) wherein Z is halogen or trifluoromethanesulfonate:

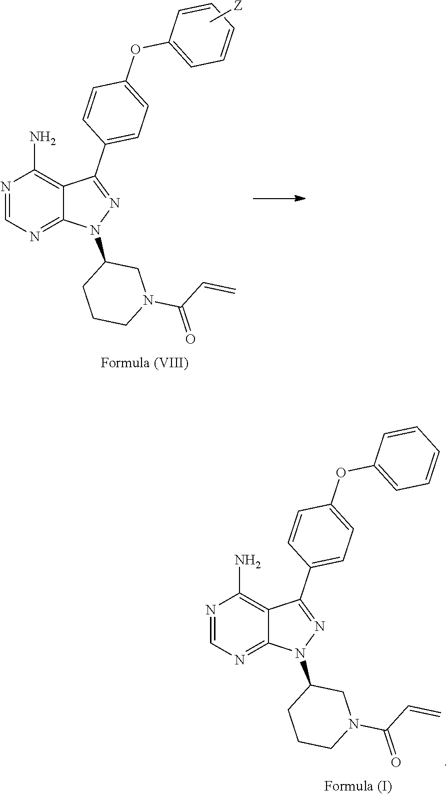

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (IX) wherein X is a halogen or sulfonate, with a compound of Formula (X) wherein Y is an alkyltin, boronic acid or boronic ester:

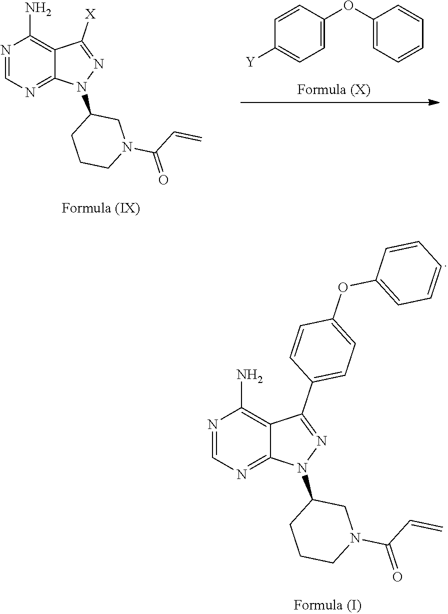

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (XI) wherein Y is an alkyltin, boronic acid or boronic ester, with a compound of Formula (XII) wherein X is a halogen or sulfonate:

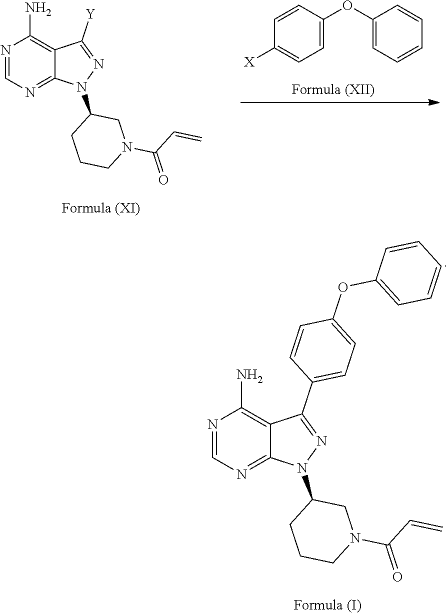

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (XIa) wherein PG is H or a protecting group such as CO—W, W is alkyl, halogenated alkyl, such as CF₃, alkoxy, dialkylamino (NR¹R², wherein R¹ and R² are each independently C₁-C₆alkyl), with a compound of Formula (XIIa) wherein X is a halogen or sulfonate, OSO₂R, B(OR)₂, N₂⁺ (diazonium), or SO₂R, wherein R is independently C₁-C₆alkyl, C₁-C₆haloalkyl, aryl or arylalkyl:

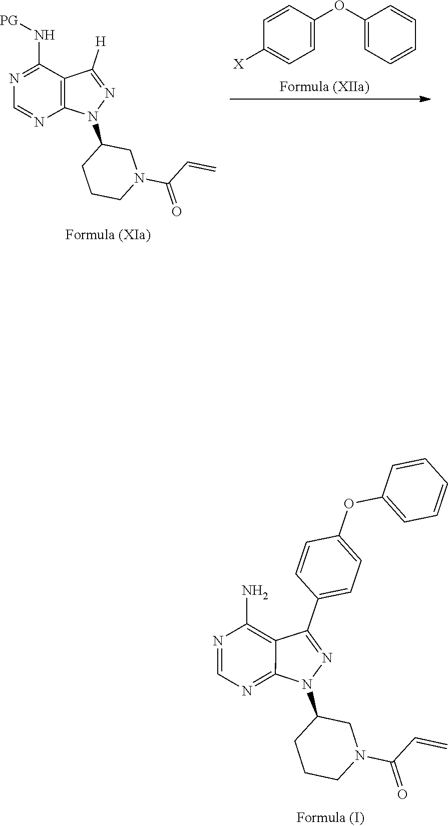

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reducing the compound of Formula (XIII):

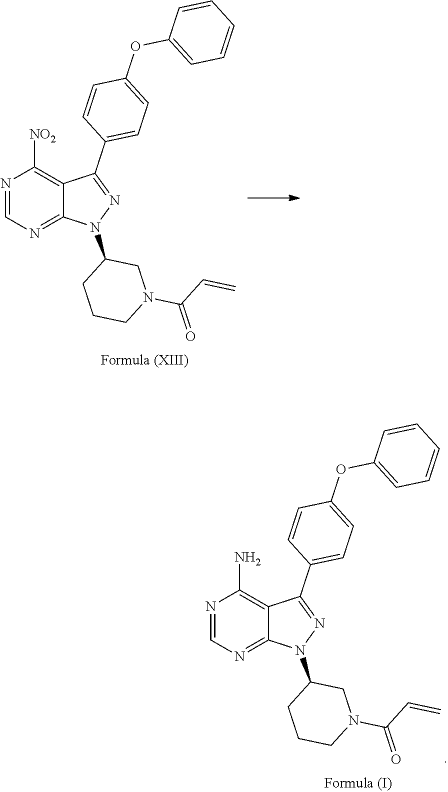 [SMILES: C=CC(=O)N1CCC[C@@H](n2nc(-c3ccc(Oc4ccccc4)cc3)c3c([N+](=O)[O-])ncnc32)C1 ; C=CC(=O)N1CCC[C@@H](n2nc(-c3ccc(Oc4ccccc4)cc3)c3c(N)ncnc32)C1]

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising deprotecting a compound of Formula (XIV), wherein PG is an amino protecting group:

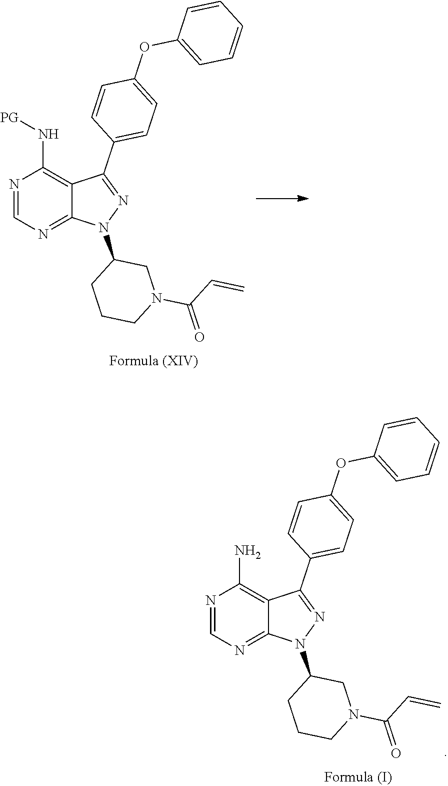

In a further embodiment described herein, the process comprises deprotecting the compound of Formula (XIV), wherein PG is benzyl, benzyl carbamate, or t-butyl carbamate.

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting the compound of Formula (XV) with a compound of Formula (XVI) wherein X is hydroxy, halogen, or sulfonate:

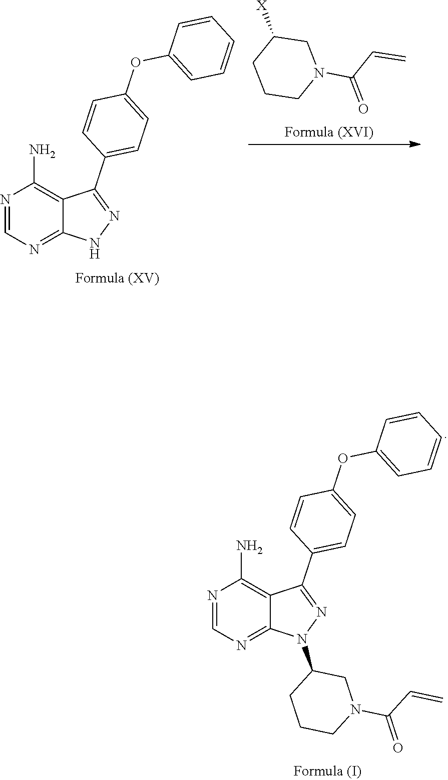

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising the β-elimination of a compound of Formula (XVII) wherein L is a leaving group:

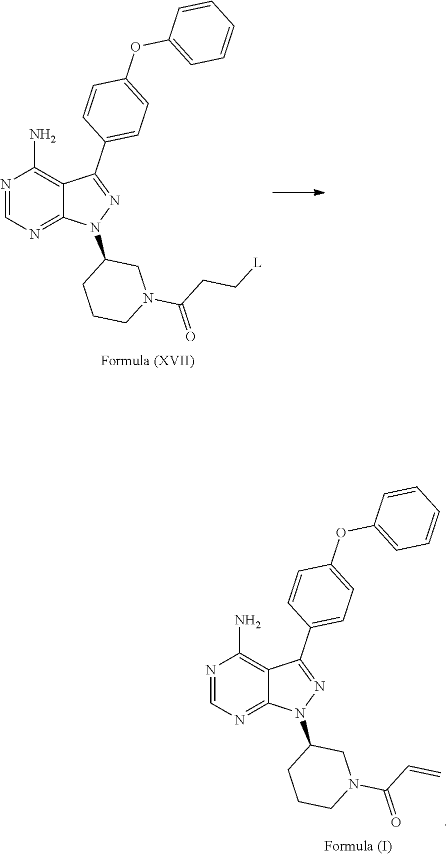

In a further embodiment described herein, the process comprises the β-elimination of a compound of Formula (XVII), wherein L is halogen, hydroxy, alkoxy, methanesulfonate, or trifluoromethanesulfonate.

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising the β-elimination of a compound of Formula (XVIII) wherein L is a leaving group:

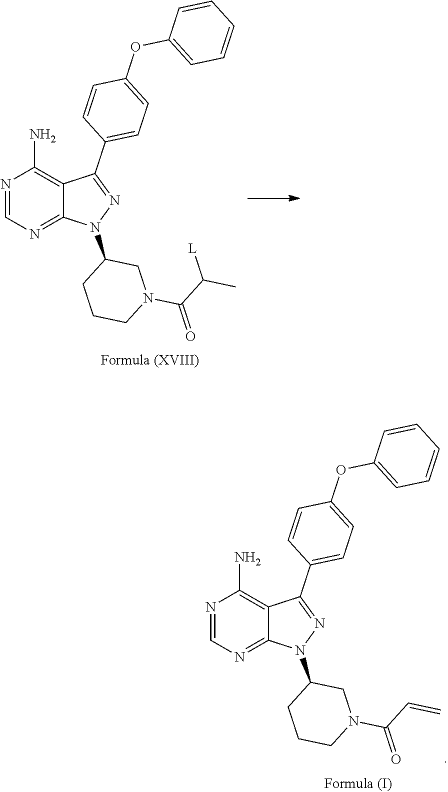

In a further embodiment described herein, the process comprises the β-elimination of a compound of Formula (XVIII), wherein L is halogen, hydroxy, alkoxy, methanesulfonate, or trifluoromethanesulfonate.

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising the reaction of a compound of Formula (XIX) wherein X is a halogen, with triphenylphosphine and formaldehyde:

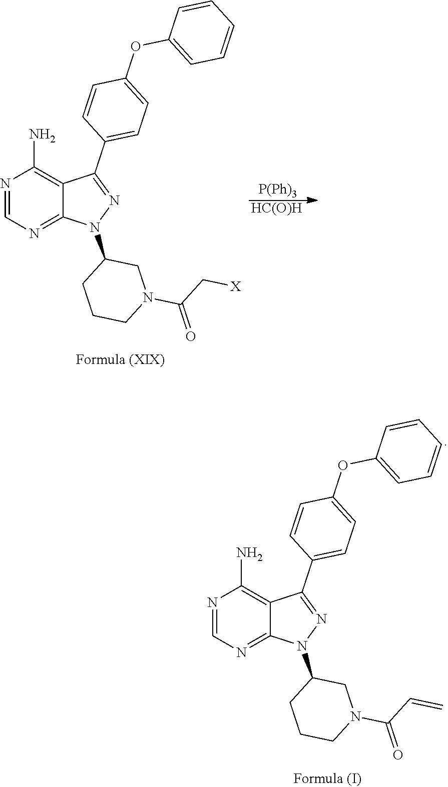

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (XX) wherein X is halogen, with a compound of Formula (XXI) wherein Y is an alkyltin, boronic acid or boronic ester:

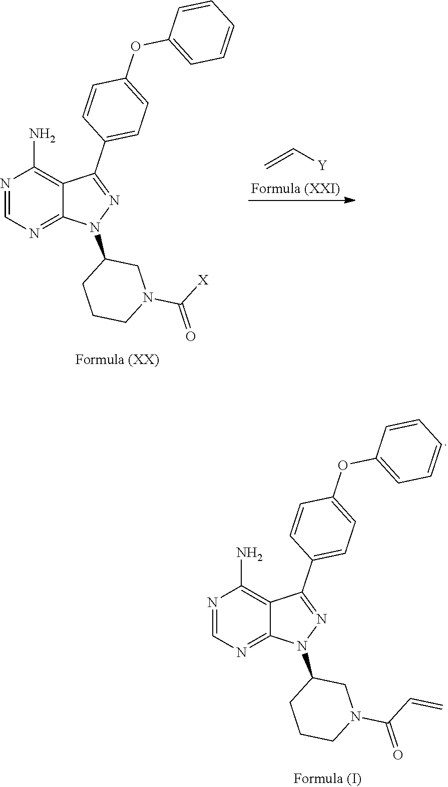

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising the hydrogenation of a compound of Formula (XXII):

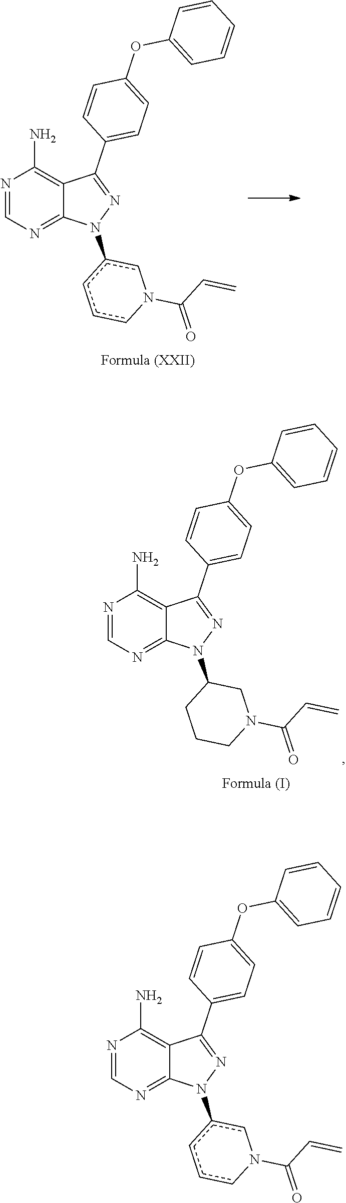

wherein Formula (XXII) represents a compound of formula (XXIIa)-(XXIIg):

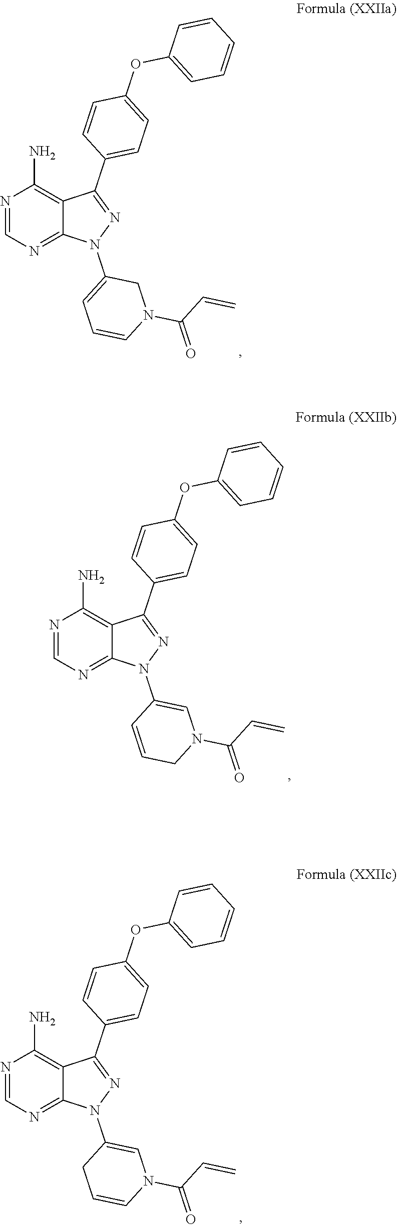 [SMILES: C=CC(=O)N1C=CC=C(n2nc(-c3ccc(Oc4ccccc4)cc3)c3c(N)ncnc32)C1 ; C=CC(=O)N1C=C(n2nc(-c3ccc(Oc4ccccc4)cc3)c3c(N)ncnc32)C=CC1 ; C=CC(=O)N1C=CCC(n2nc(-c3ccc(Oc4ccccc4)cc3)c3c(N)ncnc32)=C1]

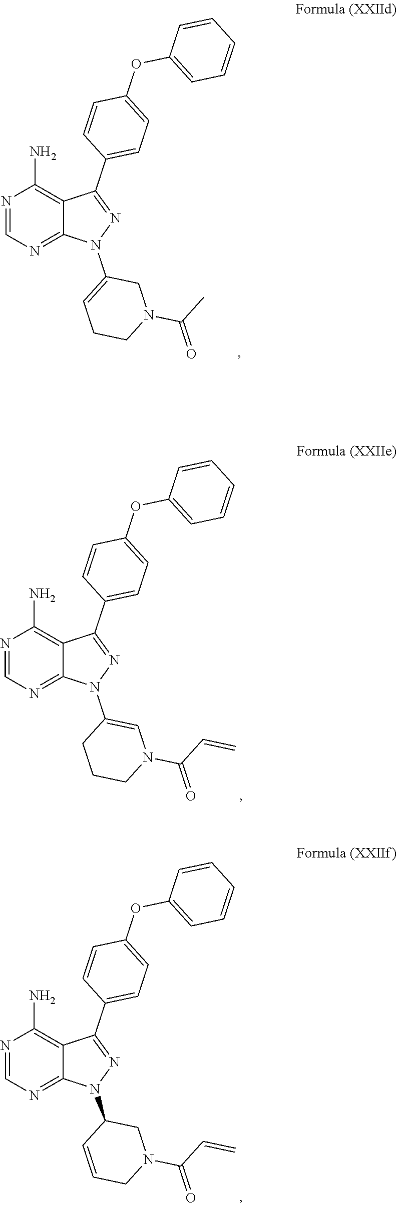 [SMILES: CC(=O)N1CCC=C(n2nc(-c3ccc(Oc4ccccc4)cc3)c3c(N)ncnc32)C1 ; C=CC(=O)N1C=C(n2nc(-c3ccc(Oc4ccccc4)cc3)c3c(N)ncnc32)CCC1 ; C=CC(=O)N1CC=C[C@@H](n2nc(-c3ccc(Oc4ccccc4)cc3)c3c(N)ncnc32)C1]

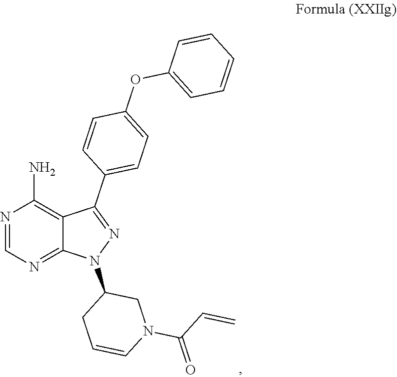 [SMILES: C=CC(=O)N1C=CC[C@@H](n2nc(-c3ccc(Oc4ccccc4)cc3)c3c(N)ncnc32)C1]

or a combination thereof.

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising the condensation of the compound of Formula (XXIII) with formamide, ammonium formate, trimethyl orthoformate with ammonia, or formamidine or a salt thereof, such as hydrochloride or acetate salt:

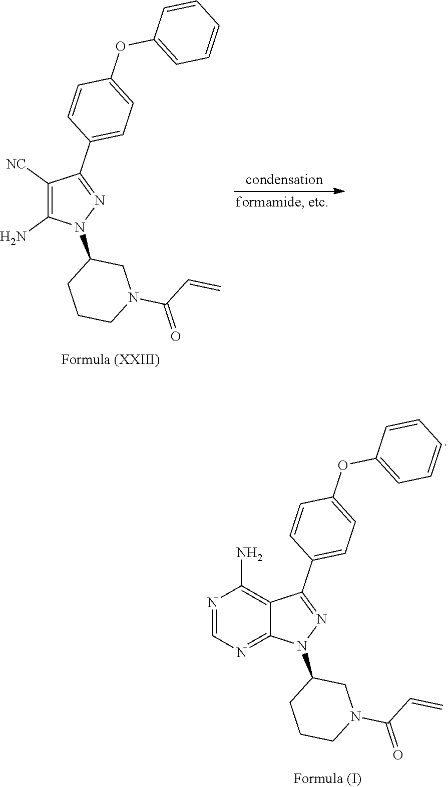 [SMILES: C=CC(=O)N1CCC[C@@H](n2nc(-c3ccc(Oc4ccccc4)cc3)c(C#N)c2N)C1 ; C=CC(=O)N1CCC[C@@H](n2nc(-c3ccc(Oc4ccccc4)cc3)c3c(N)ncnc32)C1]

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (XXIV) wherein X is a leaving group, with the compound of Formula (XXV):

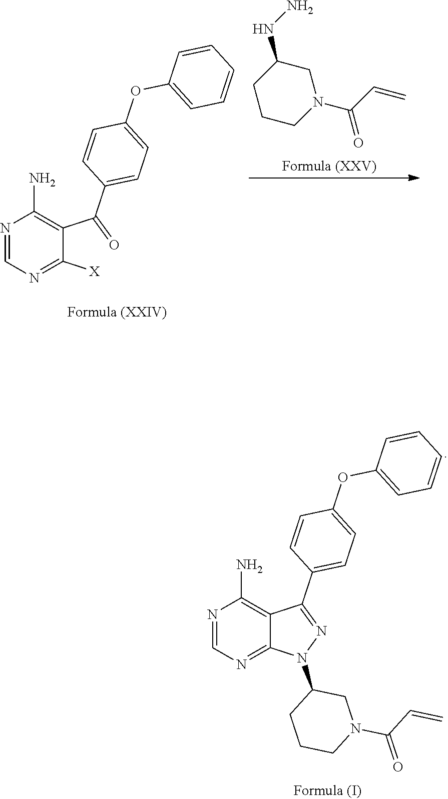

In some embodiments of Formula (XXIV), X is halogen, hydroxy, alkoxy, —P(═O)R⁶ (wherein R⁶ is independently OH, OR⁷ (R⁷ is alkyl) or halo (e.g., Cl)), methanesulfonate or trifluoromethanesulfonate. In some embodiments of Formula (XXIV), X is halogen, hydroxy, alkoxy, or trifluoromethanesulfonate. In some embodiments of Formula (XXIV), X is halogen. In some embodiments of Formula (XXIV), X is dichlorophosphate.

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (XXVI) wherein X is a leaving group, such as halogen or sulfonate, with acrylamide:

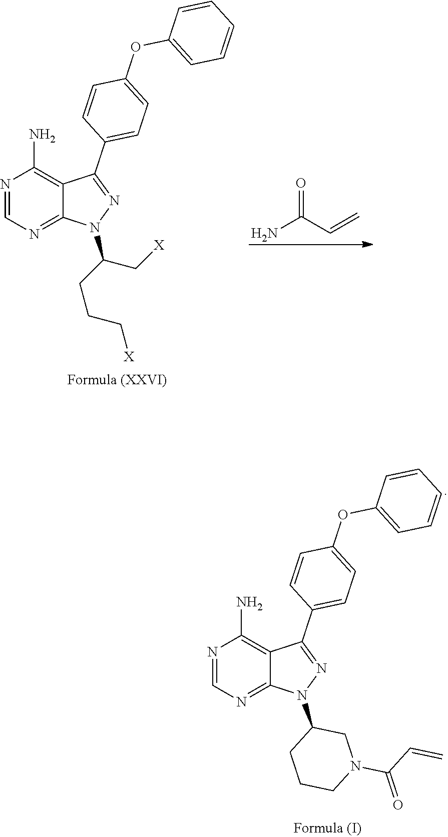

In some embodiments of Formula (XXVI), X is halogen, hydroxy, alkoxy, —P(═O)R⁶ (wherein R⁶ is independently OH, OR⁷ (R⁷ is alkyl) or halo (e.g., Cl)), methanesulfonate or trifluoromethanesulfonate. In some embodiments of Formula (XXVI), X is halogen, hydroxy, alkoxy, or trifluoromethanesulfonate. In some embodiments of Formula (XXVI), X is halogen. In some embodiments of Formula (XXVI), X is dichlorophosphate.

In another aspect, described herein, is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (XXVII) with a compound of Formula (XXVIII), wherein X is a leaving group such as hydroxy, alkoxy, halogen, sulfonate or dialkoxy-phosphoryl (P(═O)(OR⁴)₂ (each R⁴ is independently alkyl, e.g., Me or Et)):

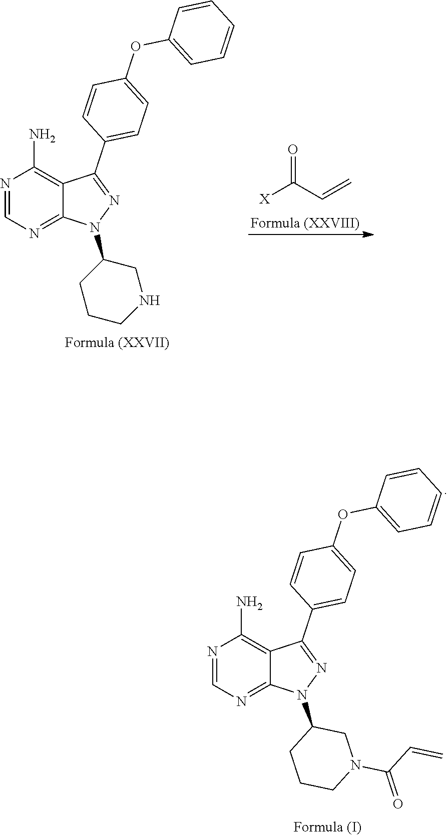

In some embodiments, X is other than Cl.

In another aspect, provided are intermediates used in any of the above processes.

### INCORPORATION BY REFERENCE

All publications and patent applications mentioned in this specification are herein incorporated by reference to the extent applicable and relevant.

### BRIEF DESCRIPTION OF THE DRAWINGS

FIG. 1 depicts the ¹H NMR of Compound XVII-1.

FIG. 2 depicts ¹³C the NMR of Compound XVII-1.

FIGS. 3, 4 and 5 depict the NMR NOE (Nuclear Overhauser Effect) of Compound XVII-1.

FIGS. 6, 7, 8 and 9 depict the NMR HMBC (Heteronuclear Multiple-bond Correlation Spectroscopy) of Compound XVII-1.

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, may be branched, straight chain, or cyclic. Depending on the structure, an alkyl group can be a monoradical or a diradical (i.e., an alkylene group). The alkyl group could also be a “lower alkyl” having 1 to 6 carbon atoms.

As used herein, C₁-Cₓ includes C₁-C₂, C₁-C₃ . . . C₁-Cₓ.

The “alkyl” moiety may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, isobutyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

An “alkoxy” group refers to a (alkyl)O— group, where alkyl is as defined herein.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “halo” or, alternatively, “halogen” or “halide” means fluoro, chloro, bromo and iodo.

A “sulfonate” group refers to a —OS(═O)₂—R, wherein R is optionally substituted alky or optionally substituted aryl.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono- and di-substituted amino groups, and the protected derivatives thereof. By way of example an optional substituents may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each Rₛ is independently selected from H, (substituted or unsubstituted C₁-C₄alkyl), (substituted or unsubstituted C₃-C₆cycloalkyl), heteroaryl, or heteroalkyl.

The term “leaving group” refers to an atom or a chemical moiety that departs as stable species taking with it the bonding electrons in bond cleavage, e.g., in substitution or elimination reactions. Leaving groups are generally known in the art. Examples of leaving groups include, but are not limited to, halogen such as Cl, Br, and I, sulfonate such as tosylate, methanesulfonate (mesylate), trifluoromethanesulfonate (triflate), hydroxyl, alkoxy, phosphate, substituted phosphate or dialkoxy-phosphoryl. In some embodiments, leaving group is OSO₂R, B(OR)₂, N₂⁺ (diazonium), or SO₂R, wherein R is independently C₁-C₆alkyl, C₁-C₆haloalkyl, aryl or arylalkyl.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

The term “Bruton's tyrosine kinase,” as used herein, refers to Bruton's tyrosine kinase from Homo sapiens, as disclosed in, e.g., U.S. Pat. No. 6,326,469 (GenBank Accession No. NP_000052).

The term “isolated,” as used herein, refers to separating and removing a component of interest from components not of interest. Isolated substances can be in either a dry or semi-dry state, or in solution, including but not limited to an aqueous solution. The isolated component can be in a homogeneous state or the isolated component can be a part of a pharmaceutical composition that comprises additional pharmaceutically acceptable carriers and/or excipients. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

The term “substantially” when referred to herein, e.g. in the context of “substantially isolated form”, refers to greater than 50% or, in an embodiment, greater than 80%, such as greater than 90% or, in a further embodiment, greater than 95% (e.g. greater than 98%). For instance, in the context of an isolated form, this means greater than 50% (by weight) of the material isolated contains the desired material or, in the other embodiments, greater than 80%, 90%, 95% or 98% (by weight).

### Synthetic Routes

In some embodiments, the processes described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fischer Scientific (Fischer Chemicals), and Acros Organics.

In further embodiments, the processes described herein employtechniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Greene and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein.

The products of the reactions may be isolated and purified, if desired, using conventional techniques, including, but not limited to, filtration, distillation, crystallization, chromatography and the like. Such materials may be characterized using conventional means, including physical constants and spectral data.

Compounds described herein may be prepared using the synthetic methods described herein as a single isomer or a mixture of isomers.

In some embodiments, the processes described herein are as outlined in the following schemes.

In one aspect, provided is a process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), which process comprises reacting a compound of Formula (II) with the compound of Formula (III) wherein X is a halogen or —B(OR⁵)₂, wherein each R⁵ is independently H or alkyl, or two R⁵ together with the B and O atoms to which they are attached form a cyclical structure:

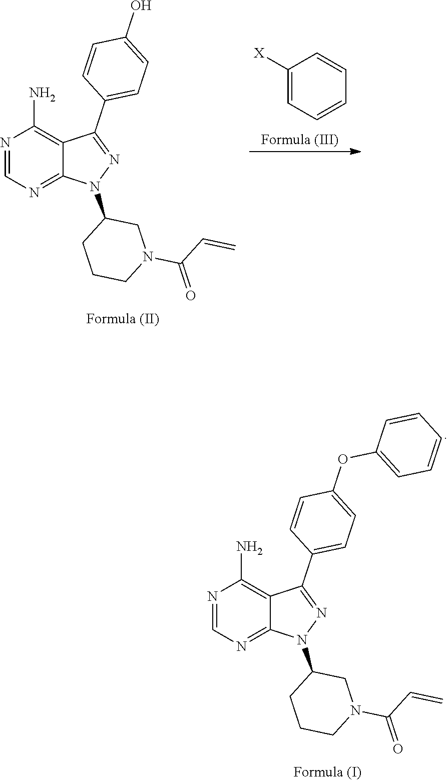

In some embodiments, the compound of Formula (II) is prepared according to Scheme 1 described below.

In a further embodiment described herein, the reacting the compound of Formula (II) with a compound of Formula (III) is in the presence of a catalyst. In some embodiments, the catalyst comprises copper, nickel, titanium or palladium, such as a salt, oxide, or complex of copper, nickel, titanium or palladium. In some embodiments, X is halogen. In some embodiments, two R⁵ together form an alkylene.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 1:

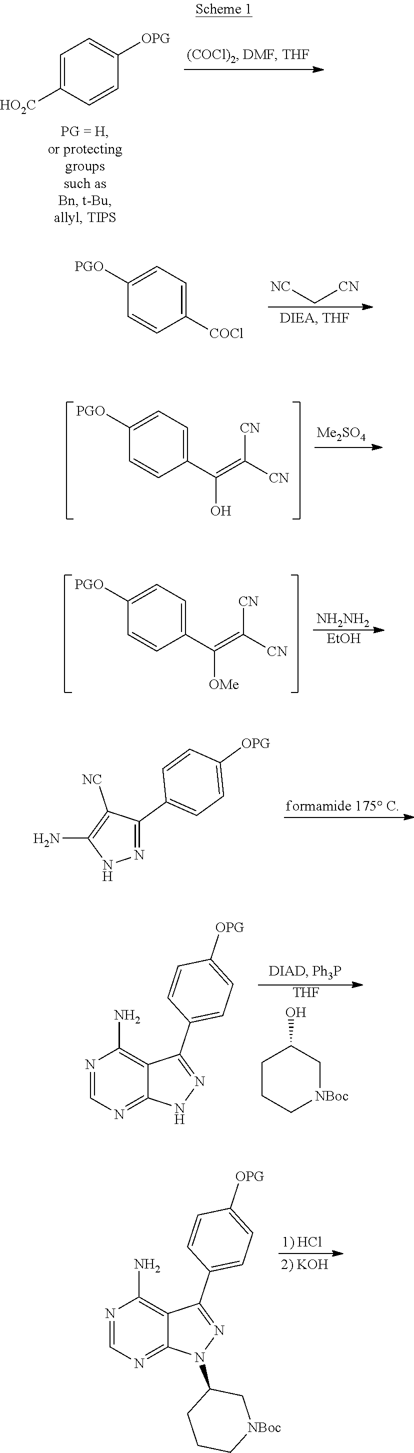

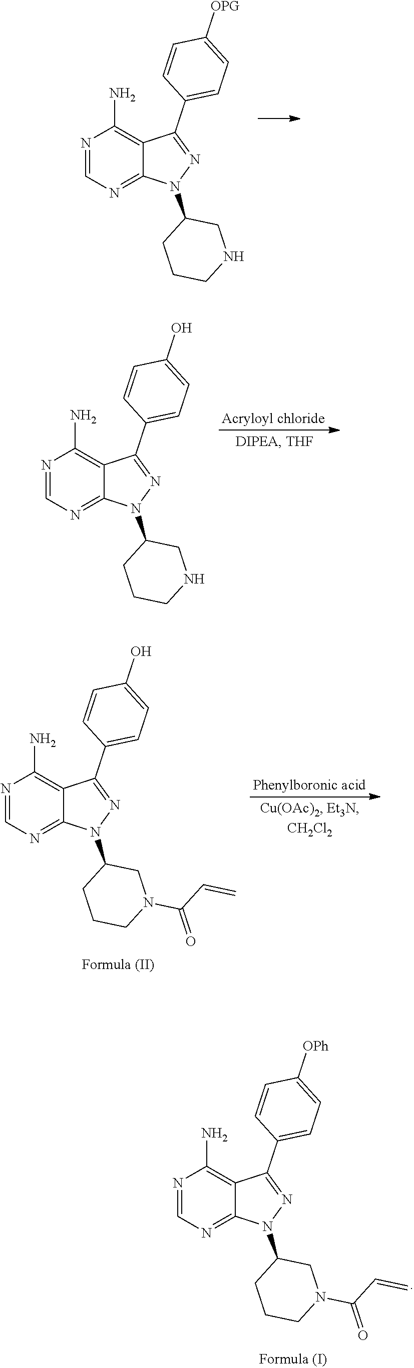

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
A) the reaction of a compound with the structure

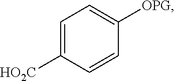

wherein PG is H or a protecting group, with oxalyl chloride in the presence of dimethylformamide (DMF) and a solvent to produce a compound with the structure

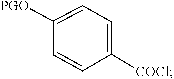

B) followed by the reaction of the compound with the structure

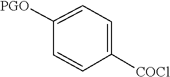

with malononitrile in the presence of a base and a solvent to produce a compound with the structure

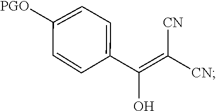

C) followed by the reaction of the compound with the structure

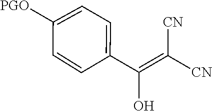

with dimethylsulfate to produce a compound with the structure

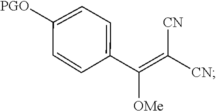

D) followed by the reaction of the compound with the structure

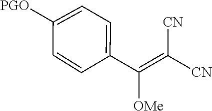

with hydrazine in the presence of a solvent to produce a compound with the structure

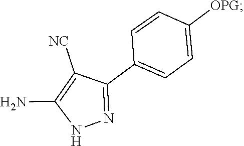

E) followed by the reaction of the compound with the structure

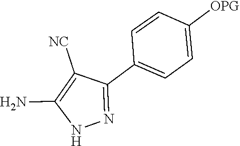

with formamide, ammonium formate, trimethyl orthoformate with ammonia, or formamidine or a salt thereof, such as hydrochloride or acetate salt, and with heating to produce a compound with the structure

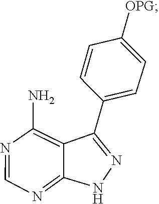

F) followed by the reaction of the compound with the structure

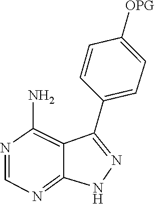

with (S)-tert-butyl 3-hydroxypiperidine-1-carboxylate, triphenyl phosphine, and diisopropyl diazodicarboxylate in the presence of a solvent to produce a compound with the structure

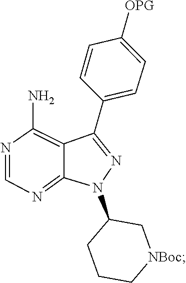

G) followed by the reaction of the compound with the structure

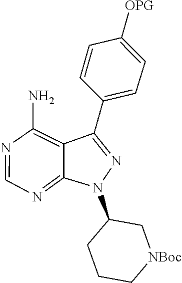

with an acid and then a base in the presence of a solvent to produce a compound with the structure

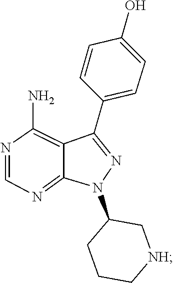 [SMILES: Nc1ncnc2c1c(-c1ccc(O)cc1)nn2[C@@H]1CCCNC1]

H) followed by the reaction of the compound with the structure

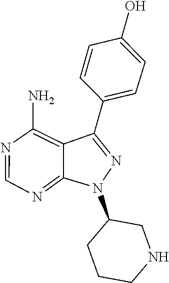 [SMILES: Nc1ncnc2c1c(-c1ccc(O)cc1)nn2[C@@H]1CCCNC1]

with a base and then acryloyl chloride in the presence of a solvent to produce a compound with the structure of Formula (II)

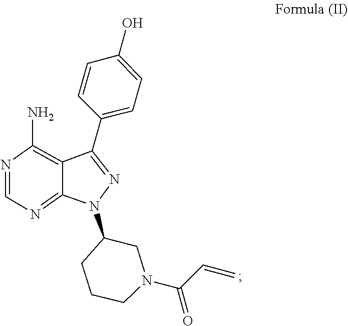 [SMILES: C=CC(=O)N1CCC[C@@H](n2nc(-c3ccc(O)cc3)c3c(N)ncnc32)C1]

G) followed by the reaction of the compound with the structure of Formula (II),

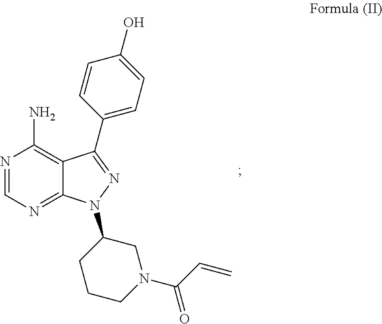 [SMILES: C=CC(=O)N1CCC[C@@H](n2nc(-c3ccc(O)cc3)c3c(N)ncnc32)C1]

with phenylboronic acid in the presence of a base, a catalyst, and a solvent to produce a compound with the structure of Formula (I),

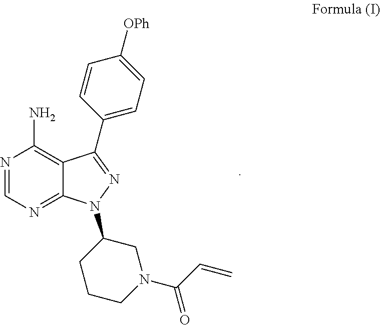 [SMILES: C=CC(=O)N1CCC[C@@H](n2nc(-c3ccc(Oc4ccccc4)cc3)c3c(N)ncnc32)C1]

In some embodiments of the process of Scheme 1, PG is H.

In some embodiments of the process of Scheme 1, PG is a protecting group, such as benzyl, t-butyl, allyl, triisopropylsilyl or tetrahydropyranyl. In some embodiments of the process of Scheme 1, PG is benzyl. In some embodiments of the process of Scheme 1, PG is t-butyl. In some embodiments of the process of Scheme 1, PG is allyl. In some embodiments of the process of Scheme 1, PG is triisopropylsilyl. In some embodiments of the process of Scheme 1, PG is tetrahydropyranyl.

In some embodiments of the process of Scheme 1, the base is selected from MOH, M₂CO₃, and MHCO₃ wherein M is selected from lithium, sodium, potassium, and cesium; 1,8-diazabicyclo[5.4.0]undec-7-ene (DBU), R₁R₂R₃N wherein R₁, R₂, and R₃ are each independently C₁-C₆alkyl. In some embodiments of the process of Scheme 1, the base is MOH. In some embodiments of the process of Scheme 1, the base is NaOH. In some embodiments of the process of Scheme 1, the base is KOH. In some embodiments of the process of Scheme 1, the base is 1,8-diazabicyclo[5.4.0]undec-7-ene (DBU). In some embodiments of the process of Scheme 1, the base is R₁R₂R₃N wherein R₁, R₂, and R₃ are each independently C₁-C₆alkyl. In some embodiments of the process of Scheme 1, the base is R₁R₂R₃N wherein R₁, R₂, and R₃ are each ethyl. In some embodiments of the process of Scheme 1, the base is R₁R₂R₃N wherein R₁ and R₂ are isopropyl and R₃ is ethyl.

In some embodiments of the process of Scheme 1, the acid is an inorganic acid. In some embodiments of the process of Scheme 1, the acid is an inorganic acid wherein the inorganic acid is hydrochloric acid, hydrobromic acid, sulfuric acid, phosphoric acid, or metaphosphoric acid. In some embodiments of the process of Scheme 1, the acid is hydrochloric acid. In some embodiments of the process of Scheme 1, the acid is hydrobromic acid. In some embodiments of the process of Scheme 1, the acid is sulfuric acid. In some embodiments of the process of Scheme 1, the acid is phosphoric acid. In some embodiments of the process of Scheme 1, the acid is metaphosphoric acid.

In some embodiments of the process of Scheme 1, the acid is an organic acid. In some embodiments of the process of Scheme 1, the acid is an organic acid, wherein the organic acid is acetic acid, propionic acid, hexanoic acid, cyclopentanepropionic acid, glycolic acid, pyruvic acid, lactic acid, malonic acid, succinic acid, malic acid, L-malic acid, maleic acid, oxalic acid, fumaric acid, trifluoroacetic acid, tartaric acid, L-tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, or valproic acid.

In some embodiments of the process of Scheme 1, the solvent is selected from water, C₁-C₆alcohol, tetrahydrofuran, 2-methyltetrahyrofuran, toluene, dichloromethane, dichloroethane, and mixtures thereof. In some embodiments of the process of Scheme 1, the solvent is water. In some embodiments of the process of Scheme 1, the solvent is C₁-C₆alcohol. In some embodiments of the process of Scheme 1, the solvent is methanol. In some embodiments of the process of Scheme 1, the solvent is isopropanol. In some embodiments of the process of Scheme 1, the solvent is tetrahydrofuran. In some embodiments of the process of Scheme 1, the solvent is 2-methyltetrahyrofuran. In some embodiments of the process of Scheme 1, the solvent is toluene. In some embodiments of the process of Scheme 1, the solvent is dichloromethane. In some embodiments of the process of Scheme 1, the solvent is dichloroethane.

In some embodiments of the process of Scheme 1, the catalyst comprises a metal, such as copper, nickel, titanium or palladium. In some embodiments, the catalyst comprises copper, nickel, titanium or palladium. In some embodiments, the catalyst is a salt, oxide, or complex of copper, nickel, titanium or palladium. In some embodiments, the catalyst is a copper salt (e.g., copper (II) acetate) used with a base. In some embodiments, the base is an inorganic base such as MOH, M₂CO₃ (wherein M is selected from lithium, sodium, potassium, and cesium), CaCO₃, di- and tri-basic phosphates (e.g. M₃PO₄, M₂HPO₄) or bicarbonates (MHCO₃). In some embodiments, the base is an organic base, such as tri-substituted amine, pyridine or 4-dimethylaminopyridine. In some embodiments, the base is NR₁R₂R₃ wherein R₁, R₂, and R₃ are each independently C₁-C₆alkyl, such as triethylamine.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 2:

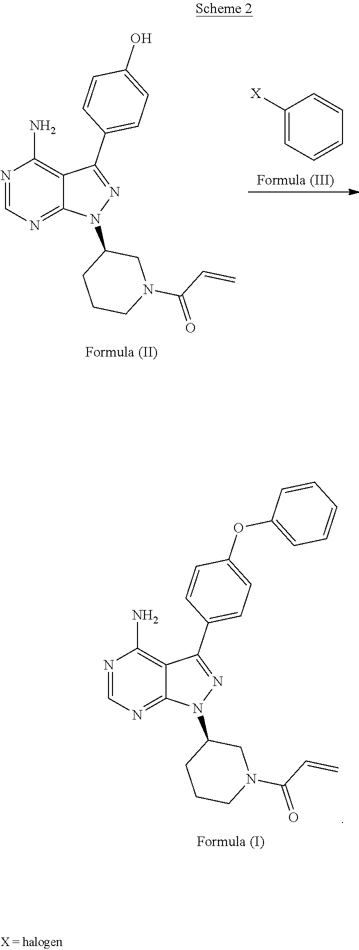

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the coupling of the compound with the structure of Formula (II),

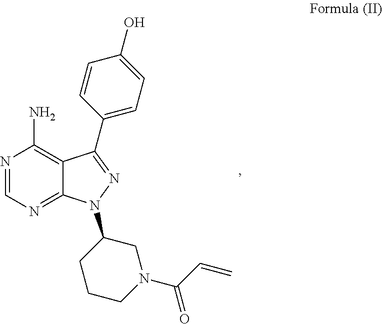 [SMILES: C=CC(=O)N1CCC[C@@H](n2nc(-c3ccc(O)cc3)c3c(N)ncnc32)C1]

with a compound with the structure of Formula (III),

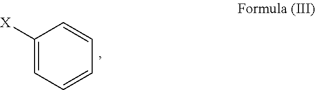

wherein X is a halogen,
   in the presence of a catalyst to produce a compound with the structure of Formula (I),

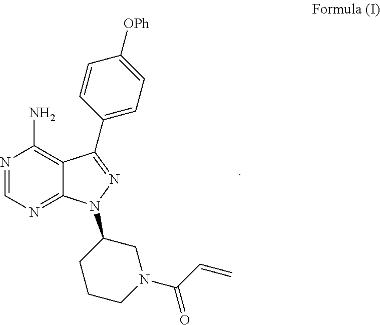 [SMILES: C=CC(=O)N1CCC[C@@H](n2nc(-c3ccc(Oc4ccccc4)cc3)c3c(N)ncnc32)C1]

In some embodiments of the process of Scheme 2, X is Cl. In some embodiments of the process of Scheme 2, X is Br. In some embodiments of the process of Scheme 2, X is I.

In some embodiments of the process of Scheme 2, the catalyst comprises a metal, such as copper, nickel, titanium or palladium. In some embodiments, the catalyst comprises copper, nickel, titanium or palladium. In some embodiments, the catalyst is a salt, oxide, or complex of copper, nickel, titanium or palladium. In some embodiments, the catalyst is a copper salt (e.g., copper (II) acetate) used with a base. In some embodiments, the base is an inorganic base such as MOH, M₂CO₃ (wherein M is selected from lithium, sodium, potassium, and cesium), CaCO₃, di- and tri-basic phosphates (e.g. M₃PO₄, M₂HPO₄) or bicarbonates (MHCO₃). In some embodiments, the base is an organic base, such as tri-substituted amine, pyridine or 4-dimethylaminopyridine. In some embodiments, the base is NR₁R₂R₃ wherein R₁, R₂, and R₃ are each independently C₁-C₆alkyl, such as triethylamine.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 3:

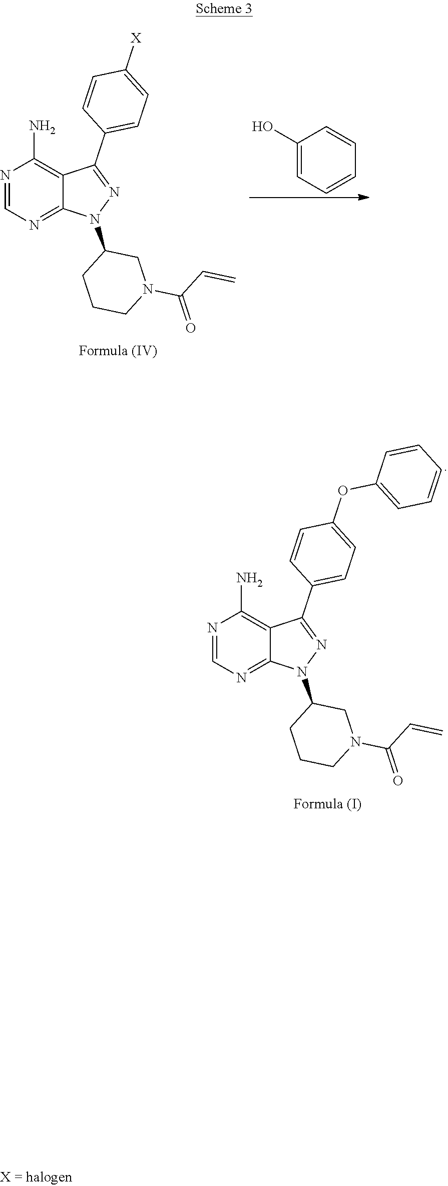

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the coupling of the compound with the structure of Formula (IV),

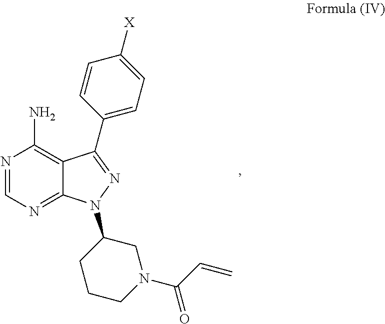

wherein X is a halogen, with phenol in the presence of copper salts to produce a compound with the structure of Formula (I),

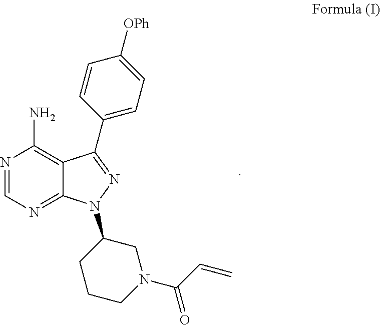 [SMILES: C=CC(=O)N1CCC[C@@H](n2nc(-c3ccc(Oc4ccccc4)cc3)c3c(N)ncnc32)C1]

In some embodiments of the process of Scheme 3, X is Cl. In some embodiments of the process of Scheme 3, X is Br. In some embodiments of the process of Scheme 3, X is I.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 4:

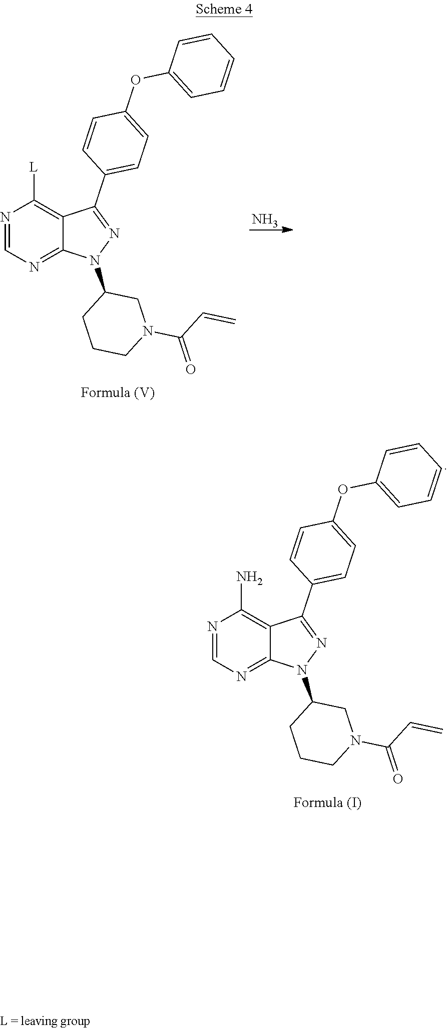

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the coupling of the compound with the structure of Formula (V),

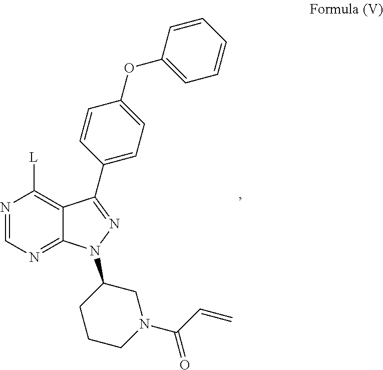

wherein L is a leaving group, such as halogen, hydroxyl, alkoxy or trifluoromethanesulfonate, in the presence of ammonia to produce a compound with the structure of Formula (I),

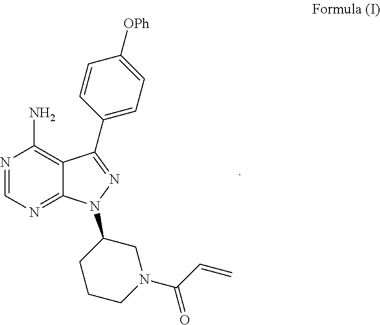 [SMILES: C=CC(=O)N1CCC[C@@H](n2nc(-c3ccc(Oc4ccccc4)cc3)c3c(N)ncnc32)C1]

In some embodiments of the process of Scheme 4, L is halogen, hydroxy, alkoxy, —P(═O)R⁶ (wherein R⁶ is independently OH, OR⁷ (R⁷ is alkyl) or halo (e.g., Cl), methanesulfonate or trifluoromethanesulfonate. In some embodiments of the process of Scheme 4, L is halogen. In some embodiments of the process of Scheme 4, L is hydroxy. In some embodiments of the process of Scheme 4, L is alkoxy. In some embodiments of the process of Scheme 4, L is methoxy. In some embodiments of the process of Scheme 4, L is ethoxy. In some embodiments of the process of Scheme 4, L is methanesulfonate. In some embodiments of the process of Scheme 4, L is trifluoromethanesulfonate. In some embodiments of the process of Scheme 4, L is dichlorophosphate.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 5:

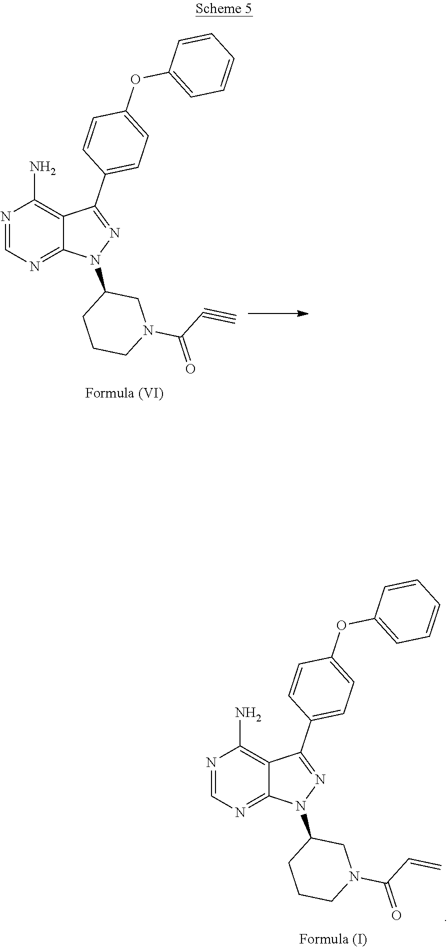 [SMILES: C#CC(=O)N1CCC[C@@H](n2nc(-c3ccc(Oc4ccccc4)cc3)c3c(N)ncnc32)C1 ; C=CC(=O)N1CCC[C@@H](n2nc(-c3ccc(Oc4ccccc4)cc3)c3c(N)ncnc32)C1]

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the reduction of the compound with the structure of Formula (VI),

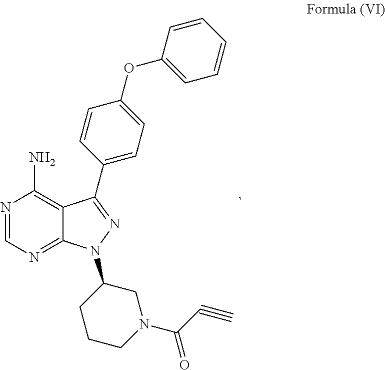 [SMILES: C#CC(=O)N1CCC[C@@H](n2nc(-c3ccc(Oc4ccccc4)cc3)c3c(N)ncnc32)C1]

to produce a compound with the structure of Formula (I),

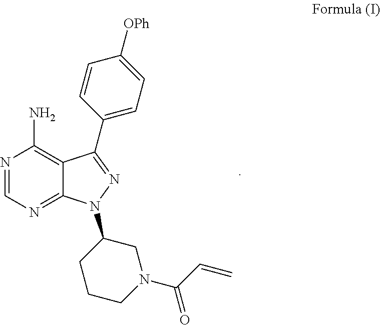 [SMILES: C=CC(=O)N1CCC[C@@H](n2nc(-c3ccc(Oc4ccccc4)cc3)c3c(N)ncnc32)C1]

In some embodiments of the process of Scheme 5, the reductive process is catalytic hydrogenation.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 6:

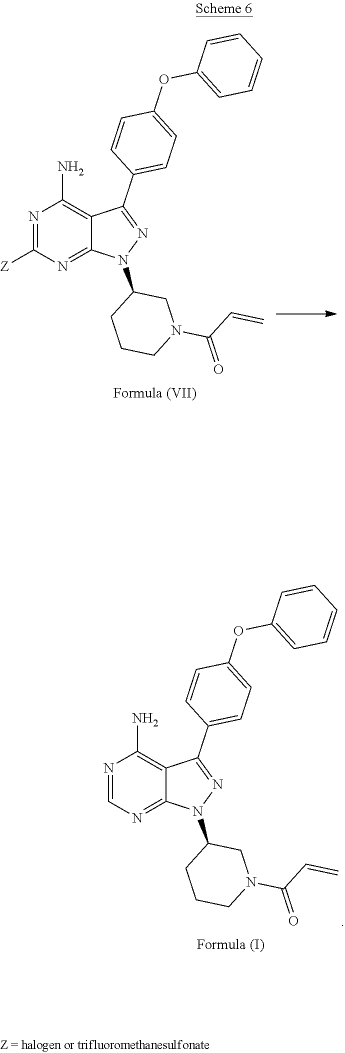

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the reduction of a compound with the structure of Formula (VII),

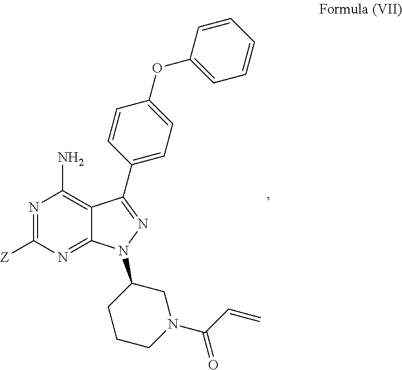

wherein Z is a halogen or trifluoromethanesulfonate, to produce a compound with the structure of Formula (I),

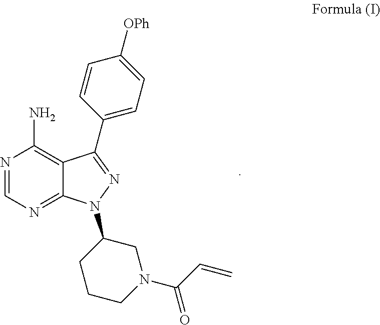 [SMILES: C=CC(=O)N1CCC[C@@H](n2nc(-c3ccc(Oc4ccccc4)cc3)c3c(N)ncnc32)C1]

In some embodiments of the process of Scheme 6, Z is halogen. In some embodiments of the process of Scheme 6, Z is trifluoromethanesulfonate.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 7:

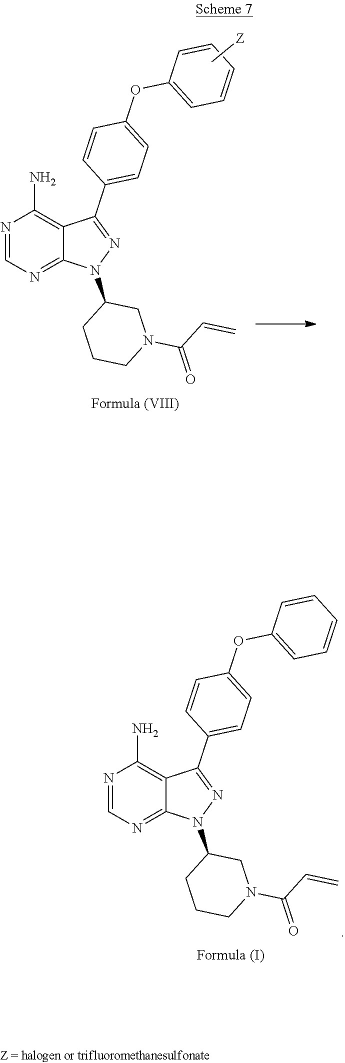

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the reduction of a compound with the structure of Formula (VIII),

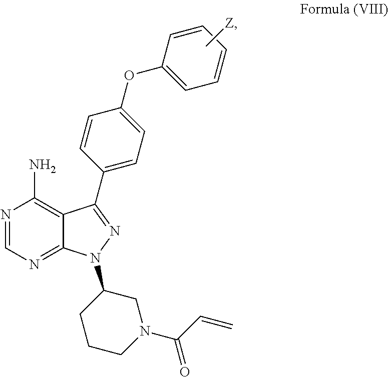

wherein Z is a halogen or trifluoromethanesulfonate, to produce a compound with the structure of Formula (I),

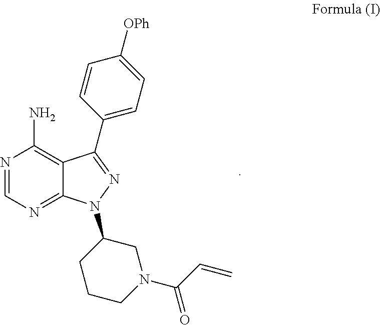 [SMILES: C=CC(=O)N1CCC[C@@H](n2nc(-c3ccc(Oc4ccccc4)cc3)c3c(N)ncnc32)C1]

In some embodiments of the process of Scheme 7, Z is halogen. In some embodiments of the process of Scheme 7, Z is trifluoromethanesulfonate.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 8:

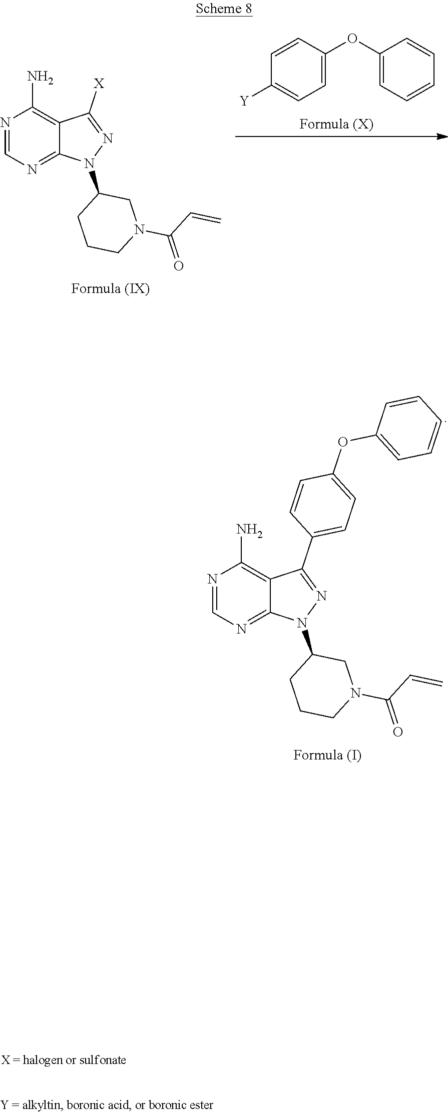

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the coupling of the compound with the structure of Formula (IX),

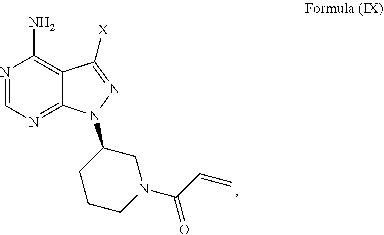

wherein X is halogen or sulfonate, with a compound with the structure of Formula (X),

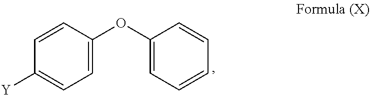

wherein Y is an alkyltin, boronic acid, or boronic ester, to produce a compound with the structure of Formula (I),

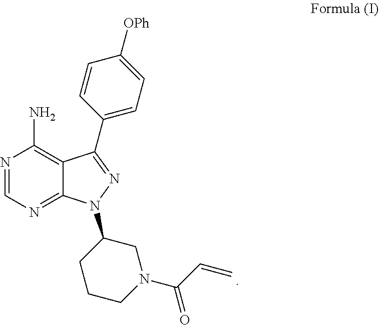 [SMILES: C=CC(=O)N1CCC[C@@H](n2nc(-c3ccc(Oc4ccccc4)cc3)c3c(N)ncnc32)C1]

In some embodiments of the process of Scheme 8, X is halogen. In some embodiments of the process of Scheme 8, X is a sulfonate. In some embodiments of the process of Scheme 8, X is trifluoromethanesulfonate. In some embodiments of the process of Scheme 8, Y is an alkyltin. In some embodiments of the process of Scheme 8, Y is a boronic acid. In some embodiments of the process of Scheme 8, Y is a boronic ester, such as —B(OR′R″), wherein R′ and R″ are each independently alkyl or R′ and R″ together form an alkylene or substituted alkylene.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 9:

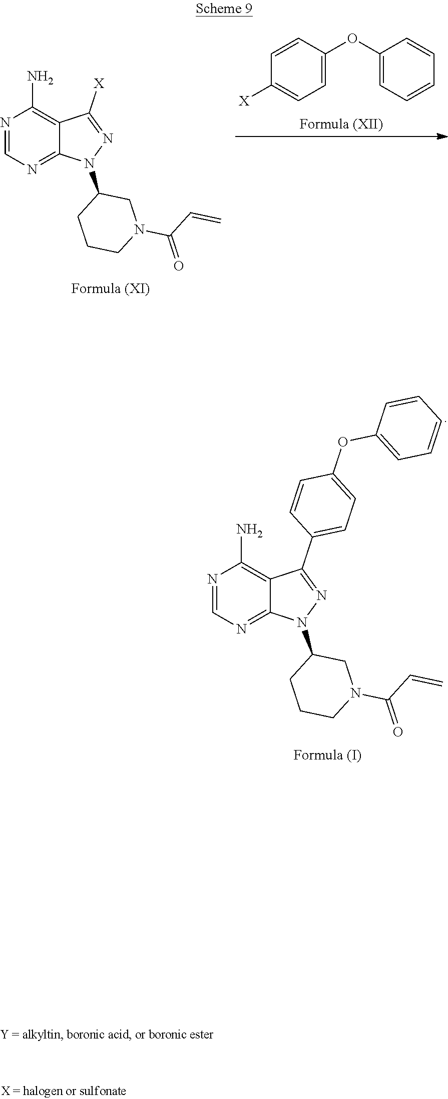

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the coupling of the compound with the structure of Formula (XI),

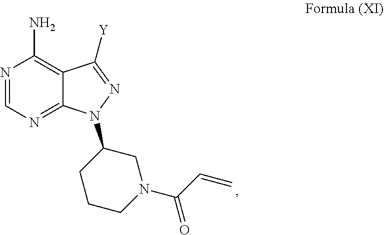

wherein Y is an alkyltin, boronic acid, or boronic ester, with a compound with the structure of Formula (XII),

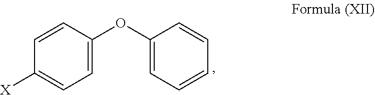

wherein X is halogen or sulfonate, to produce a compound with the structure of Formula (I),

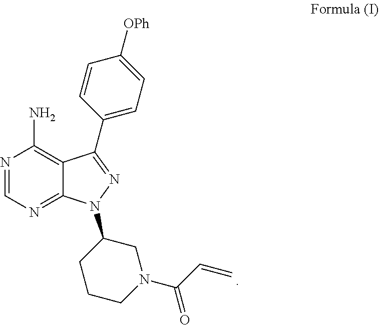 [SMILES: C=CC(=O)N1CCC[C@@H](n2nc(-c3ccc(Oc4ccccc4)cc3)c3c(N)ncnc32)C1]

In some embodiments of the process of Scheme 9, X is halogen. In some embodiments of the process of Scheme 9, X is a sulfonate. In some embodiments of the process of Scheme 9, X is trifluoromethanesulfonate. In some embodiments of the process of Scheme 9, Y is an alkyltin. In some embodiments of the process of Scheme 9, Y is a boronic acid. In some embodiments of the process of Scheme 9, Y is a boronic ester, such as —B(OR′R″), wherein R′ and R″ are each independently alkyl or R′ and R″ together form an alkylene or substituted alkylene.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 10:

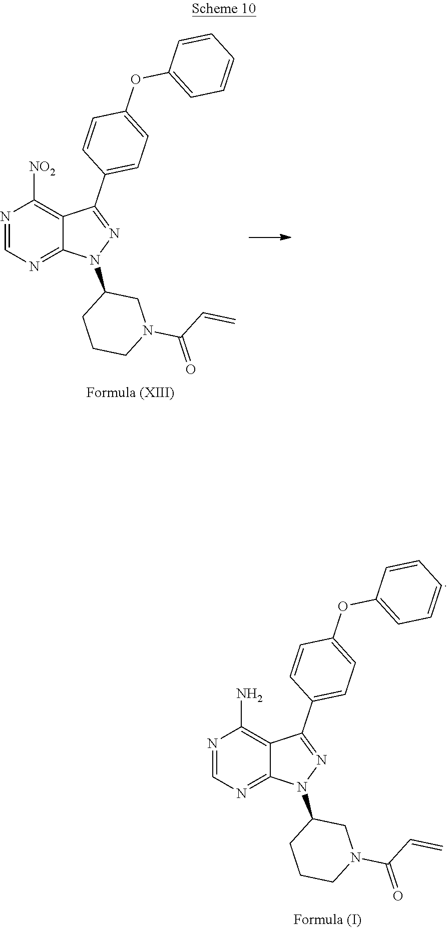 [SMILES: C=CC(=O)N1CCC[C@@H](n2nc(-c3ccc(Oc4ccccc4)cc3)c3c([N+](=O)[O-])ncnc32)C1 ; C=CC(=O)N1CCC[C@@H](n2nc(-c3ccc(Oc4ccccc4)cc3)c3c(N)ncnc32)C1]

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the reduction of the compound with the structure of Formula (XIII),

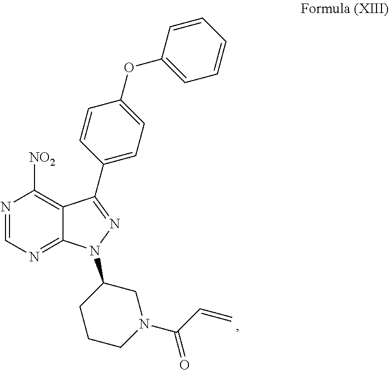 [SMILES: C=CC(=O)N1CCC[C@@H](n2nc(-c3ccc(Oc4ccccc4)cc3)c3c([N+](=O)[O-])ncnc32)C1]

to produce a compound with the structure of Formula (I)

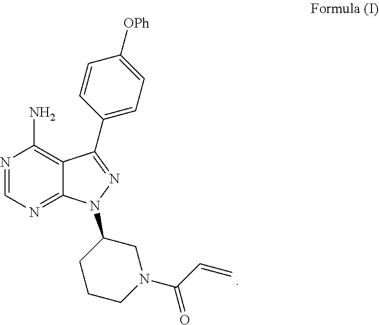 [SMILES: C=CC(=O)N1CCC[C@@H](n2nc(-c3ccc(Oc4ccccc4)cc3)c3c(N)ncnc32)C1]

In some embodiments, the reduction of the compound with the structure of Formula (XIII) to a compound with the structure of Formula (I) proceed via an intermediate compound with the structure of Formula (XIIIa):

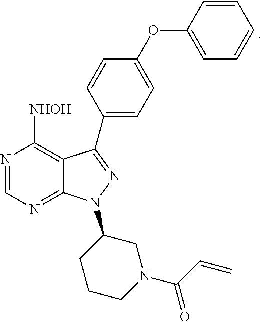

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the reduction of the compound with the structure of Formula (XIIIa),

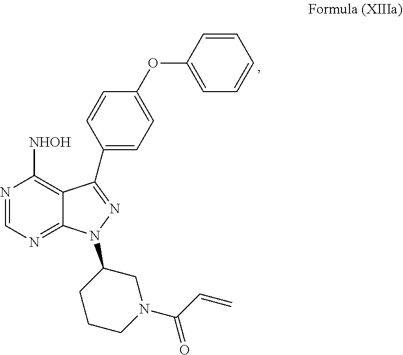

to produce a compound with the structure of Formula (I)

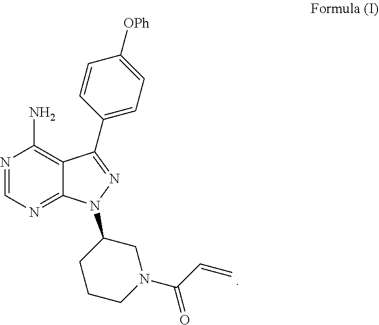 [SMILES: C=CC(=O)N1CCC[C@@H](n2nc(-c3ccc(Oc4ccccc4)cc3)c3c(N)ncnc32)C1]

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 11:

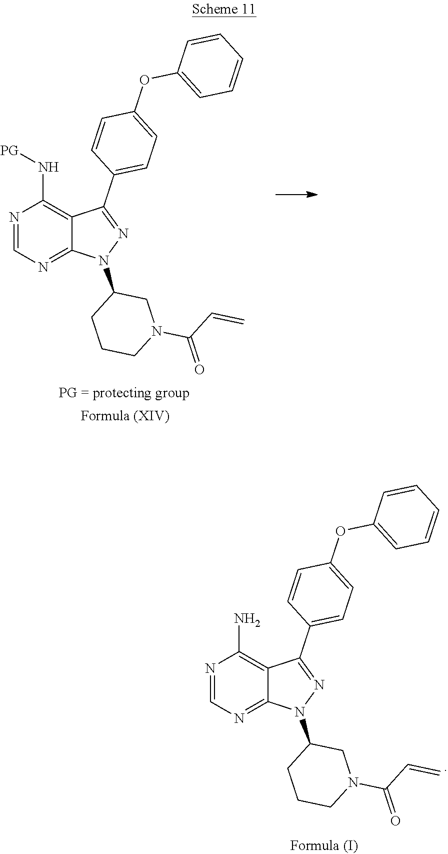

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the deprotection of a compound with the structure of Formula (XIV),

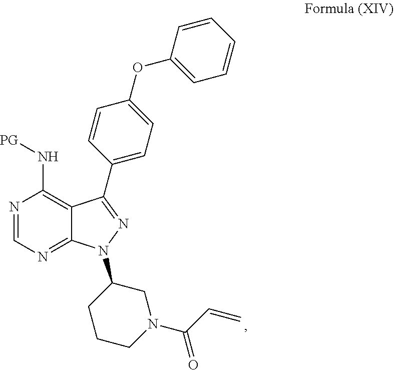

wherein PG is a protecting group, to produce a compound with the structure of Formula (I),

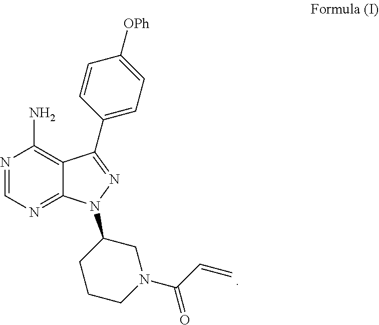 [SMILES: C=CC(=O)N1CCC[C@@H](n2nc(-c3ccc(Oc4ccccc4)cc3)c3c(N)ncnc32)C1]

In some embodiments of the process of Scheme 11, the protecting group is benzyl, benzyl carbamate, or t-butyl carbamate. In some embodiments of the process of Scheme 11, the protecting group is benzyl. In some embodiments of the process of Scheme 11, the protecting group is benzyl carbamate. In some embodiments of the process of Scheme 11, the protecting group is t-butyl carbamate.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 12:

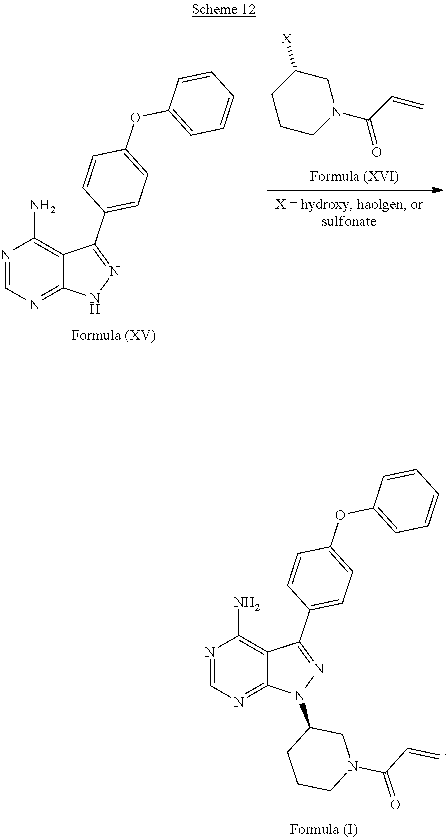

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the coupling of the compound with the structure of Formula (XV),

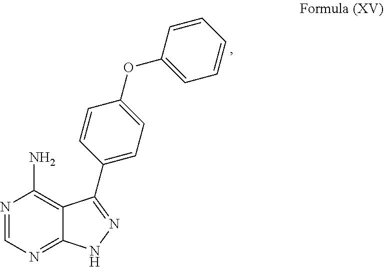 [SMILES: Nc1ncnc2[nH]nc(-c3ccc(Oc4ccccc4)cc3)c12]

with a compound with the structure of Formula (XVI),

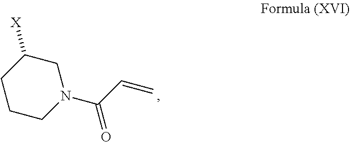

wherein X is hydroxy, halogen or sulfonate, to produce a compound with the structure of Formula (I),

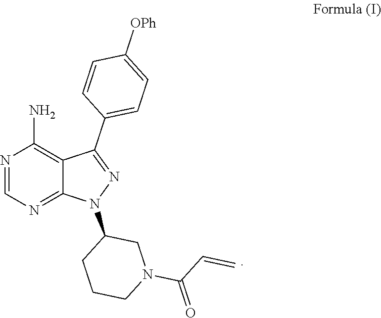 [SMILES: C=CC(=O)N1CCC[C@@H](n2nc(-c3ccc(Oc4ccccc4)cc3)c3c(N)ncnc32)C1]

In some embodiments of the process of Scheme 12, X is hydroxy, halogen or sulfonate. In some embodiments of the process of Scheme 12, X is halogen. In some embodiments of the process of Scheme 12, X is a sulfonate. In some embodiments of the process of Scheme 12, X is methanesulfonate. In some embodiments of the process of Scheme 12, X is trifluoromethanesulfonate.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 13:

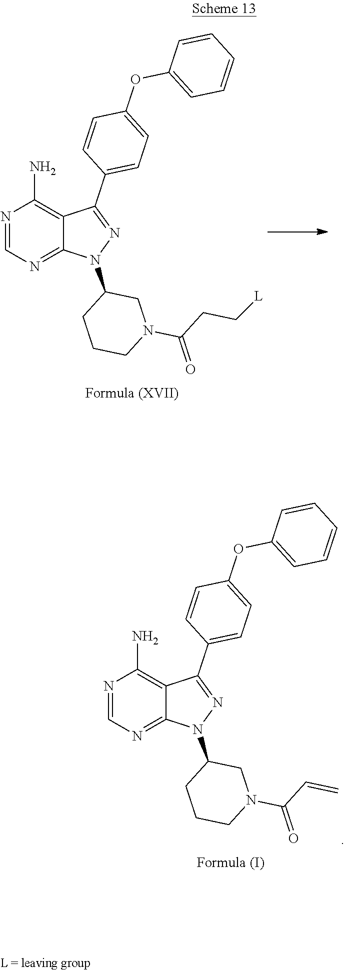

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the β-elimination of a compound with the structure of Formula (XVII),

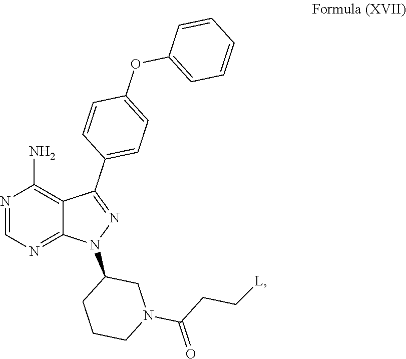

wherein L is a leaving group, to produce a compound with the structure of Formula (I),

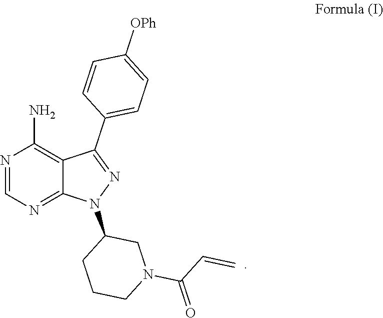 [SMILES: C=CC(=O)N1CCC[C@@H](n2nc(-c3ccc(Oc4ccccc4)cc3)c3c(N)ncnc32)C1]

In some embodiments of the process of Scheme 13, the leaving group is halogen, hydroxy, alkoxy, methanesulfonate or trifluoromethanesulfonate. In some embodiments of the process of Scheme 13, the leaving group is halogen. In some embodiments of the process of Scheme 13, the leaving group is hydroxy. In some embodiments of the process of Scheme 13, the leaving group is alkoxy. In some embodiments of the process of Scheme 13, the leaving group is trifluoromethanesulfonate.

In some embodiments, the compound of Formula (XVII) is a compound of Formula (XVII-1), and the process comprises β-elimination of the compound of Formula (XVII-1),

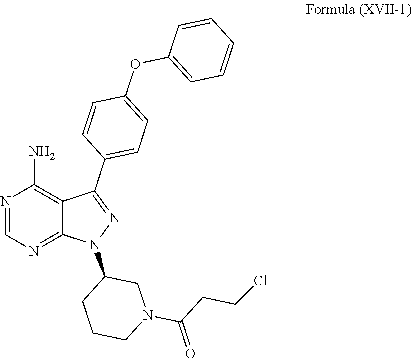 [SMILES: Nc1ncnc2c1c(-c1ccc(Oc3ccccc3)cc1)nn2[C@@H]1CCCN(C(=O)CCCl)C1]

or a pharmaceutically acceptable salt thereof.

The process comprising β-elimination of a compound with the structure of Formula (XVII), such as the compound with the structure of Formula (XVII-1), may be referred to as the “elimination process”.

In a further embodiment, there is also provided a compound of Formula (XVII), e.g., a compound of Formula (XVII-1), (as such) or a pharmaceutically acceptable salt thereof. In particular, such a compound is in a substantially isolated form and/or in a substantially purified form (for example, a HPLC purity of greater than 90%, e.g. greater than 95%).

The compound of formula (XVII) may be prepared by reaction of a compound of formula (XVII-A),

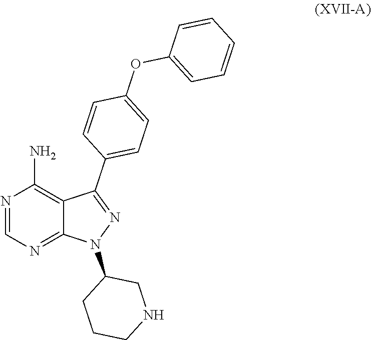 [SMILES: Nc1ncnc2c1c(-c1ccc(Oc3ccccc3)cc1)nn2[C@@H]1CCCNC1]

or a pharmaceutically acceptable salt thereof,
with L¹-C(O)—CH₂CH₂L or a salt thereof, wherein L¹ is a leaving group, such as halogen or trifluoromethanesulfonate, which process may also be referred to as the “acylation process”.

In some embodiments, L and L¹ are the same. In some embodiments, L and L¹ are different provided that the group L¹-C(O) is more reactive than CH₂L.

In another embodiment, the compound of formula (XVII-1) may be prepared by reaction of a compound of formula (XVII-A),

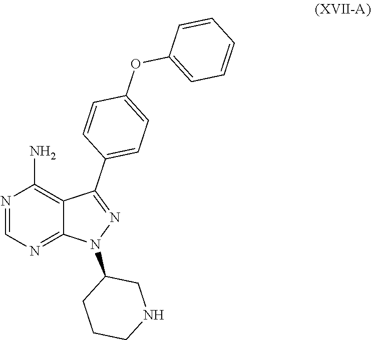 [SMILES: Nc1ncnc2c1c(-c1ccc(Oc3ccccc3)cc1)nn2[C@@H]1CCCNC1]

or a pharmaceutically acceptable salt thereof,
with L¹-C(O)—CH₂CH₂Cl or a salt thereof, wherein L¹ is a leaving group, such as halogen or trifluoromethanesulfonate. In some embodiments, the compound L¹-C(O)—CH₂CH₂Cl is 3-chloropropionyl chloride (i.e. Cl—C(O)—CH₂CH₂Cl).

In a further embodiment, there is provided a product obtainable by the acylation process.

The “elimination process” is an elimination reaction, which is preferably performed in the presence of base. Any suitable base may be employed, for example an organic or inorganic base. It is preferably a non-nucleophilic base that is suitable for the elimination reaction (i.e. a strong enough base to promote the elimination; the reaction results in the production of H⁺ and Cl⁻ ions which may form an ionic bond to produce HCl). In an embodiment, an organic base is employed. Such bases that may be employed include alkoxide bases (e.g. tert-butoxides, such as potassium tert-butoxide), amine bases (e.g. trialkylamine, such as triethylamine, dimethylaminopyridine (DMAP), N-methylmorpholine, 1,4-diazabicyclo[2.2.2]octane (DABCO), 1,8-diazabicycloundec-7-ene (DBU) or the like), amide bases (e.g. LDA or LiHMDS, i.e. lithium diisopropylamide or lithium bis(trimethylsilyl)amide) or other suitable bases (or mixtures of bases). In an embodiment the base employed is an amine base such as DBU.

In order for the elimination process to progress efficiently, at least one equivalent (compared to the compound of formula XVII) of base is needed. However, in preferred embodiments, there is an excess of base equivalents employed (the base may be one base or a mixture of more than one, e.g. two, different bases). In an embodiment, there is at least about 1.5 such as about 2 equivalents of base (e.g. between about 2 and about 5 equivalents). In an embodiment, there is either 2, 4 or 5 equivalents of base (e.g. DBU) employed (compared to the compound of formula XVII). In a preferred embodiment between about 1.5 and 2.5 (e.g. about 2) equivalents of DBU base are employed. It may be seen that different bases may result in differing reaction efficiency and/or differing yields and or purity of the desired product.

The elimination process may also be allowed to react for a suitable period of time. For instance the progress of the reaction may be monitored (e.g. by thin layer chromatography) and the duration may be for a period of between about 1 hour and about 24 hours. In the embodiment where about 2 equivalents of DBU is employed, the reaction time may be between about 4 hours and about 24 hours (preferably between about 4 and 10 hours, such as between 6 and 8 hours e.g. about 7 hours).

The elimination process is, in an embodiment, performed in the presence of a suitable solvent, such as a polar aprotic solvent. Suitable solvents therefore include solvents such as THF (tetrahydrofuran) and EtOAc (ethyl acetate). The reaction conditions are therefore preferably conducted in anhydrous or inert conditions, e.g. using anhydrous solvent and performed under an inert (e.g. N₂) atmosphere.

The reaction temperature of the elimination process is preferably between about 0° C. and about 80° C., but is dependent on the base that is intended to be employed (e.g. for a lithium amide base, low temperatures such as about 0° C. are required to avoid the base deprotonating the solvent). When a type of base other than a lithium amide (or organolithium base) is employed, then the preferred temperature range is between about room temperature (e.g. about 20° C. to about 25° C.) and about 65° C. When ethyl acetate is employed as a solvent, then the preferred temperature may be between about room temperature and about 65° C. When THF is employed, the temperature of the reaction is preferably about room temperature (e.g. between about 20 and 25° C.).

The elimination process may also include the use of an additive, for instance any suitable additive that may promote the process reaction. Suitable additives may include sodium trifluoroacetate (i.e. CF₃COONa; which may be bound to three water molecules, so forming e.g. CF₃COONa.3H₂O), sodium lactate, CH₃SO₃Na, CF₃SO₃Na or CF₃SO₃Li (or the like, e.g. another suitable metal ion instead of Na/Li may be employed and the “acid” moiety may be another suitable acid). In an embodiment, the additive is sodium trifluoroacetate (i.e. CF₃COONa).

The preferred order of addition in an embodiment of the elimination process is addition of the compound of formula XVII (together with the optional solvent), which compound and solvent may be allowed to mix together (e.g. over the course of 10-15 minutes). In an embodiment, it is then preferred that the base (e.g. about 2 equivalents of DBU) is added, preferably over the course of a period of time (e.g. between 10 minutes and 4 hours, for instance about 1 or 2 hours). The reaction is then allowed to stir for a period as specified herein.

In an embodiment, the mixture obtained as a result of the elimination process is purified. Such purification may be performed in the work up stage. For example, to the mixture of the elimination process, a suitable base may be added (for example sodium carbonate, e.g. Na₂CO₃-2 equivalents 5% Na₂CO₃), for instance after the reaction mixture is transferred to another vessel, and allowed to stir for a period of time (e.g. between about 5 minutes and 4 hours, such as between about 30 minutes and 2 hours). The reaction mixture may then be worked up. For instance, the organic phase may be washed with water and/or citric acid (particularly the latter wash may be advantageous to remove impurities). The (combined) aqueous phases may then be extracted with an organic solvent (e.g. ethyl acetate) and the organic phases combined. The combined organic phases may then be pH-adjusted as desired, for example by adding a suitable base (e.g. Na₂CO₃), for instance such that the pH is adjusted to about 6-7.5.

In the acylation process, the 3-chloropropionyl chloride is in a purity of >50% (e.g. by HPLC). Hence this distinguishes from the situation where the 3-chloropropionyl chloride may incidentally be present as an impurity. The 3-chloropropionyl chloride reagent is therefore employed in a form/purity in which is can be commercially purchased (e.g. from Sigma-Aldrich).

In an embodiment, the acylation process, the compound L¹-C(O)—CH₂CH₂L, such as 3-chloropropionyl chloride, is added in a large excess. For instance, the compound of formula (XVII-A) may first be dissolved in an appropriate solvent (e.g. a polar aprotic solvent, such as THF, methyl-THF, ethyl acetate or the like), which is anhydrous. Such a reaction may be performed under an inert atmosphere, e.g. under N₂ (or another inert gas). To the mixture of compound of formula (XVII-A) and solvent, a suitable base may then be added first. L¹-C(O)—CH₂CH₂L, such as 3-chloropropionyl chloride, (for example one equivalent or less, e.g. between 0.5 and 1 equivalents compared to the compound of formula I) may then be added (for example dropwise, in order to maintain a certain reaction temperature). The remaining L¹-C(O)—CH₂CH₂L, such as 3-chloropropionyl chloride, (given that, in an embodiment, it may be employed in excess) may be diluted with the appropriate solvent that is employed in this step of the process (for instance the polar aprotic solvent mentioned above) and that may also be slowly added over the course of a period of time (e.g. 10 minutes to 2 hours), dependent on maintaining the reaction temperature. The isolation of the desired material may be performed as set out below.

In an embodiment of the acylation process, an additive may be employed in addition to the required reactants, e.g. butylated hydroxyl toluene (BHT). Such an additive (e.g. BHT) is preferably added to the reaction mixture at the outset (e.g. together with the compound of formula (XVII-A) and solvent).

In an embodiment of the acylation process, the reaction may be performed at a temperature of room temperature or below, for instance at or below about 20 to 25° C. In an embodiment, it is preferred that it is performed at below room temperature (e.g. at about 10° C.) or in an ice bath. In an embodiment, it is preferred that the addition of the 3-chloropropionyl chloride is performed at a rate so as to maintain the reaction temperature as constant as possible, for example the time durations specified herein (e.g. to maintain the temperature at about 10° C.).

Suitable bases that may be employed in the acylation process include organic and inorganic bases. When inorganic bases are employed then Schotten-Baumann conditions may be employed (e.g. a mixture of organic and aqueous phases). Suitable inorganic bases include carbonate and bicarbonate/hydrogencarbonate bases (e.g. Na₂CO₃ or NaHCO₃).

The compound of formula XVII that is prepared by the acylation process may be isolated and/or purified. The mixture of the acylation process may be worked up, for instance the aqueous phase may be separated and the organic phase may be washed (e.g. with a sodium hydrogencarbonate wash). Thereafter, two methods may be employed to isolate and/or purify (if indeed that is the intention, i.e. in an embodiment the compound of formula XVII need not be isolated/separated) to provide the compound of formula XVII in a solid form. Crystallisation may be performed for instance using a mixture of solvents as may be described hereinafter (e.g. in the examples), for instance using a mixture of a polar aprotic solvent (e.g. a solvent that may be employed in the second process of the invention) and an alkane solvent. Polar aprotic solvents that may be mentioned include Me-THF and EtOAc (methyl-tetrahydrofuran and ethyl acetate). Alkane solvents that may be mentioned include heptane (e.g. n-heptane).

In an embodiment, the compound of formula XVII need not be separated or isolated from the acylation process but may (e.g. in a preferred embodiment) be used directly in the elimination process. This may have the advantage that it is overall a process that is more efficient or more convenient. In such an embodiment, the solvent that may be employed in the acylation process may remain the same as that solvent employed directly in the elimination process. Alternatively, the solvent used in the acylation process may be switched to a different solvent before directly being used in the elimination process. In this context, “directly” refers to the compound of formula XVII being used in the acylation process without being separated, isolated and/or purified before being used in the subsequent step, i.e. the elimination process.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 14:

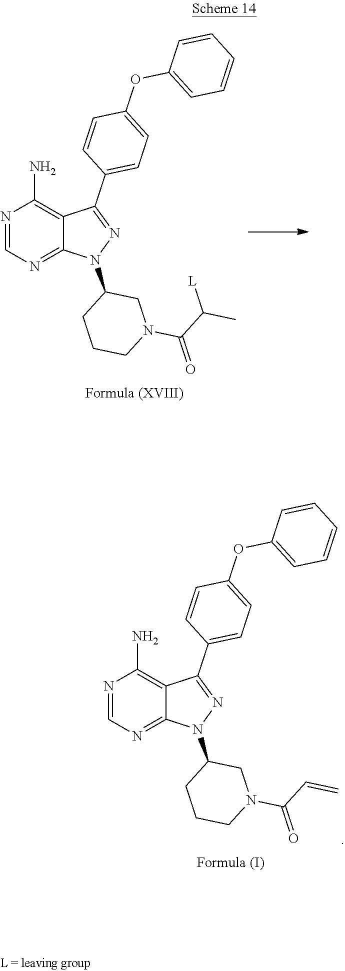

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the β-elimination of a compound with the structure of Formula (XVIII),

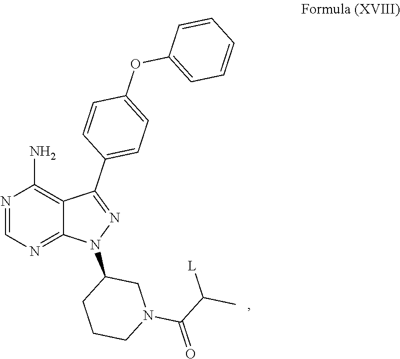

wherein L is a leaving group, to produce a compound with the structure of Formula (I),

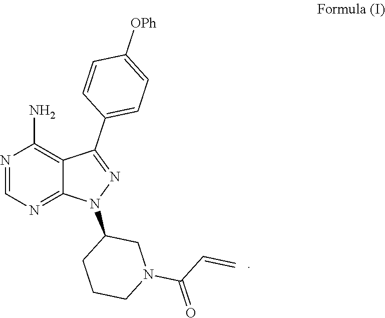 [SMILES: C=CC(=O)N1CCC[C@@H](n2nc(-c3ccc(Oc4ccccc4)cc3)c3c(N)ncnc32)C1]

In some embodiments of the process of Scheme 14, the leaving group is halogen, hydroxy, alkoxy, methanesulfonate or trifluoromethanesulfonate. In some embodiments of the process of Scheme 14, the leaving group is halogen. In some embodiments of the process of Scheme 14, the leaving group is hydroxy. In some embodiments of the process of Scheme 14, the leaving group is alkoxy. In some embodiments of the process of Scheme 14, the leaving group is trifluoromethanesulfonate.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 15:

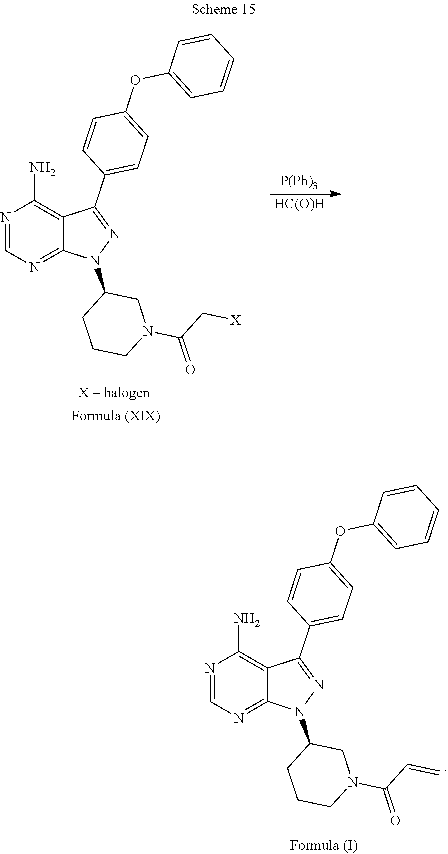

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the coupling of a compound with the structure of Formula (XIX),

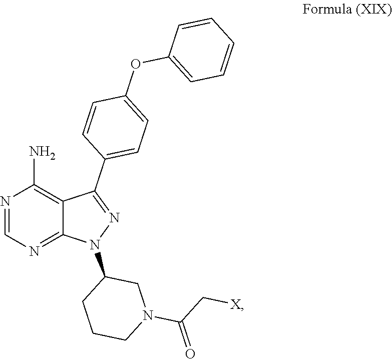

wherein X is a halogen, in the presence of triphenylphosphine and formaldehyde to produce a compound with the structure of Formula (I),

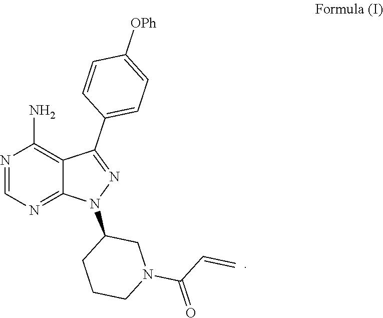 [SMILES: C=CC(=O)N1CCC[C@@H](n2nc(-c3ccc(Oc4ccccc4)cc3)c3c(N)ncnc32)C1]

In some embodiments of the process of Scheme 15, X is Cl. In some embodiments of the process of Scheme 15, X is Br.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 16:

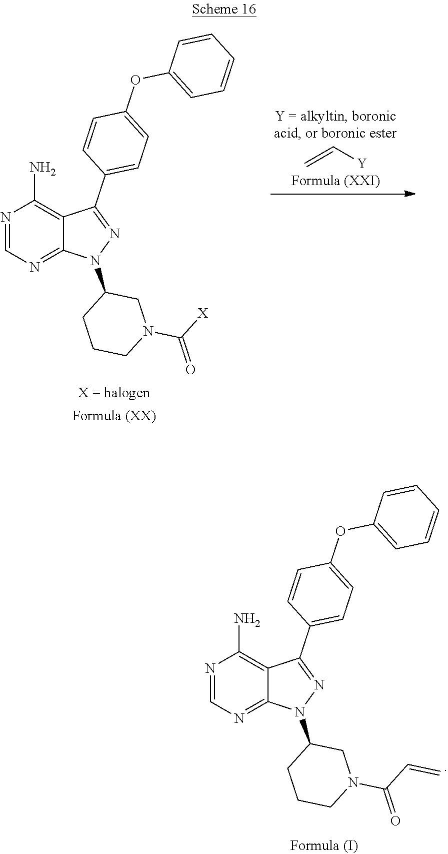

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the coupling of a compound with the structure of Formula (XX),

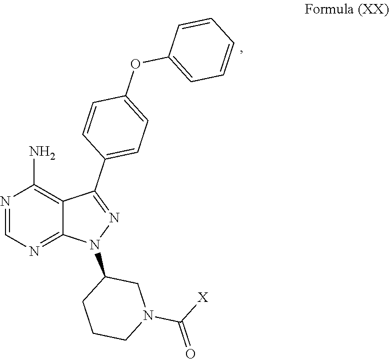

wherein X is halogen, with a compound with the structure of Formula (XXI),

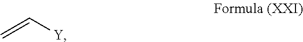

wherein Y is an alkyltin, boronic acid, or boronic ester, to produce a compound with the structure of Formula (I),

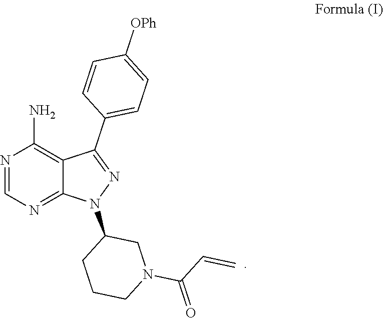 [SMILES: C=CC(=O)N1CCC[C@@H](n2nc(-c3ccc(Oc4ccccc4)cc3)c3c(N)ncnc32)C1]

In some embodiments of the process of Scheme 16, X is Cl. In some embodiments of the process of Scheme 16, Y is an alkyltin. In some embodiments of the process of Scheme 16, Y is a boronic acid. In some embodiments of the process of Scheme 16, Y is a boronic ester, such as —B(OR′R″), wherein R′ and R″ are each independently alkyl or R′ and R″ together form an alkylene or substituted alkylene.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 17:

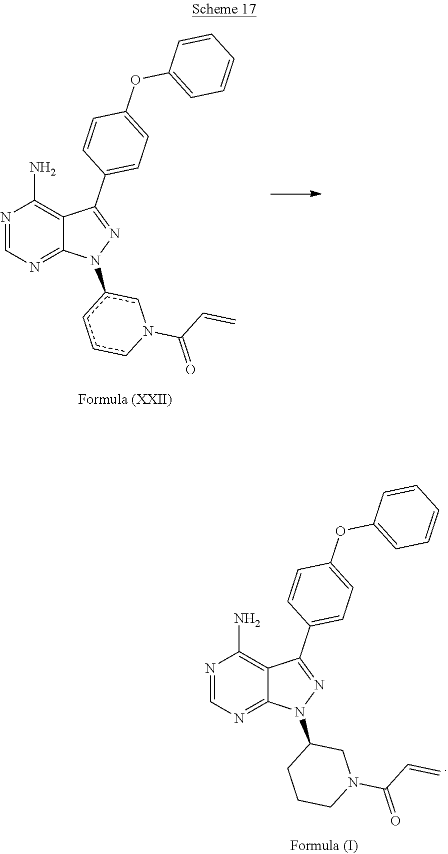

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the reduction of a compound with a structure of Formula (XXII),

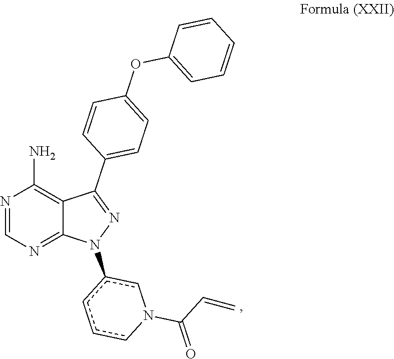

to produce a compound with the structure of Formula (I),

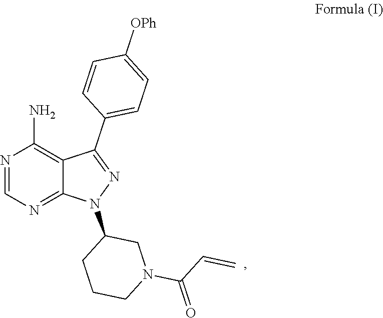 [SMILES: C=CC(=O)N1CCC[C@@H](n2nc(-c3ccc(Oc4ccccc4)cc3)c3c(N)ncnc32)C1]

wherein

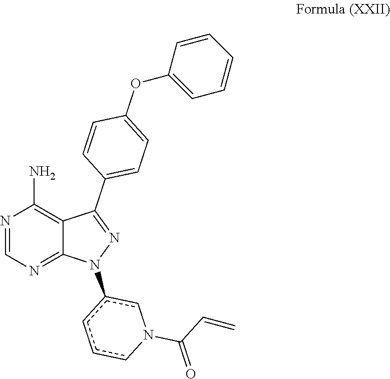

represents a compound of formula (XXIIa)-(XXIIg):

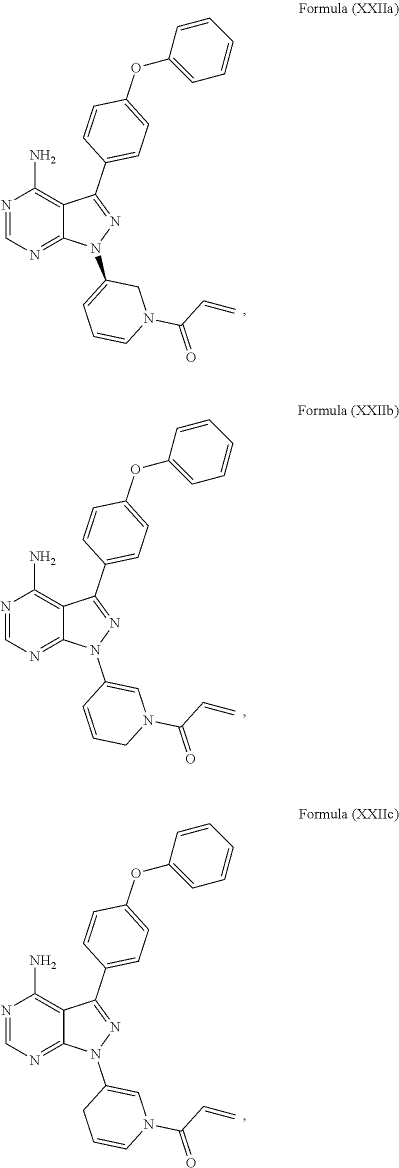

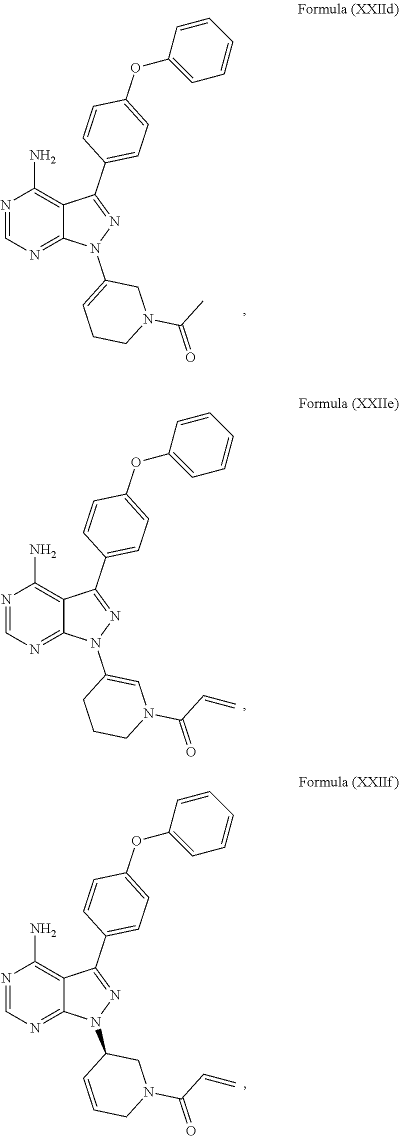

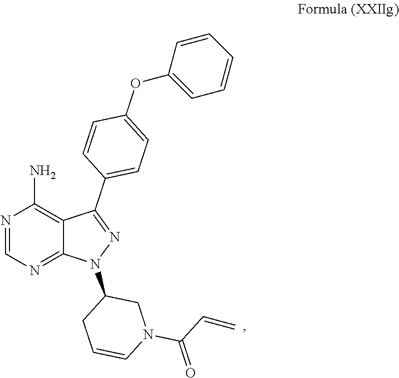 [SMILES: C=CC(=O)N1C=CC[C@@H](n2nc(-c3ccc(Oc4ccccc4)cc3)c3c(N)ncnc32)C1]

or a combination thereof.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 18:

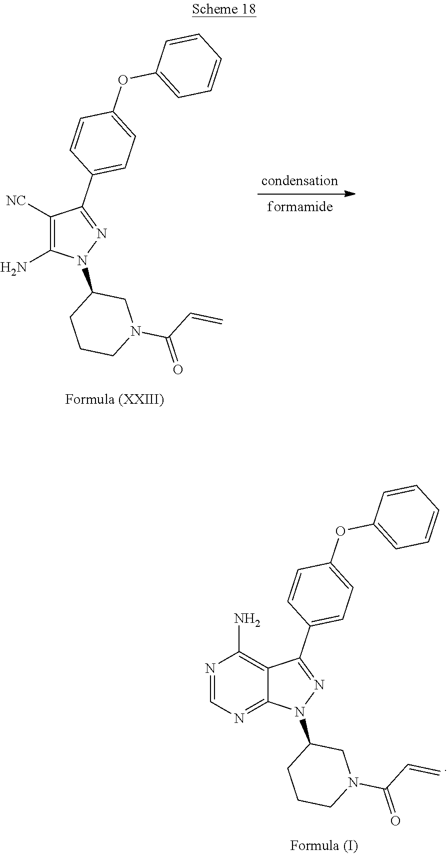 [SMILES: C=CC(=O)N1CCC[C@@H](n2nc(-c3ccc(Oc4ccccc4)cc3)c(C#N)c2N)C1 ; C=CC(=O)N1CCC[C@@H](n2nc(-c3ccc(Oc4ccccc4)cc3)c3c(N)ncnc32)C1]

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the condensation of the compound with the structure of Formula (XXIII),

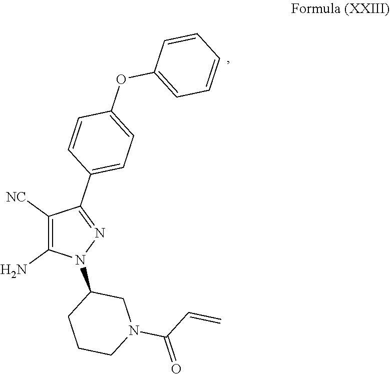 [SMILES: C=CC(=O)N1CCC[C@@H](n2nc(-c3ccc(Oc4ccccc4)cc3)c(C#N)c2N)C1]

Formula (XXIII), with formamide, ammonium formate, trimethyl orthoformate with ammonia, or formamidine or a salt thereof, such as hydrochloride or acetate salt, to produce a compound with the structure of Formula (I),

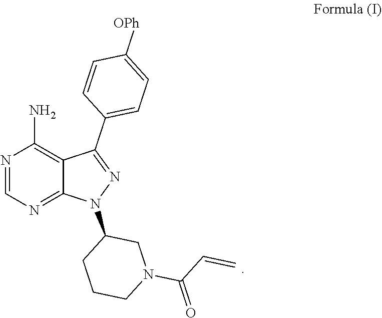 [SMILES: C=CC(=O)N1CCC[C@@H](n2nc(-c3ccc(Oc4ccccc4)cc3)c3c(N)ncnc32)C1]

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 19: Scheme 19

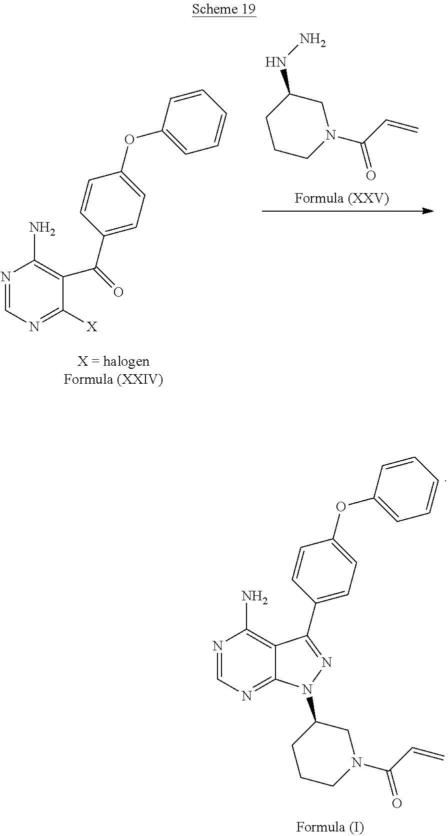

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the coupling of a compound with the structure of Formula (XXIV),

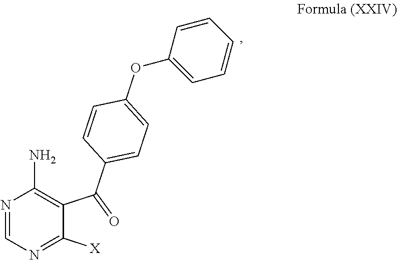

wherein X is a leaving group such as halogen, with the compound with the structure of Formula (XXV),

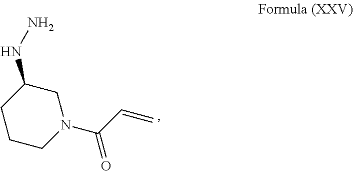

to produce a compound with the structure of Formula (I),

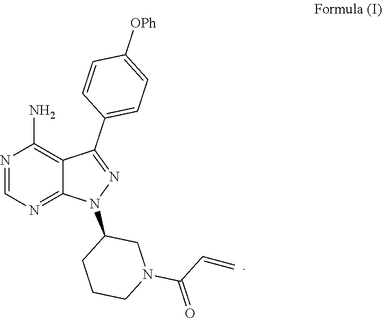 [SMILES: C=CC(=O)N1CCC[C@@H](n2nc(-c3ccc(Oc4ccccc4)cc3)c3c(N)ncnc32)C1]

In some embodiments of Formula (XXIV), X is halogen, sulfonate, phosphate, hydroxy or alkoxy. In some embodiments, X is halogen. In some embodiments, X is —P(═O)R⁶ (wherein R⁶ is independently OH, OR⁷ (R⁷ is alkyl) or halo (e.g., Cl)). In some embodiments, X is dichlorophosphate.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 20:

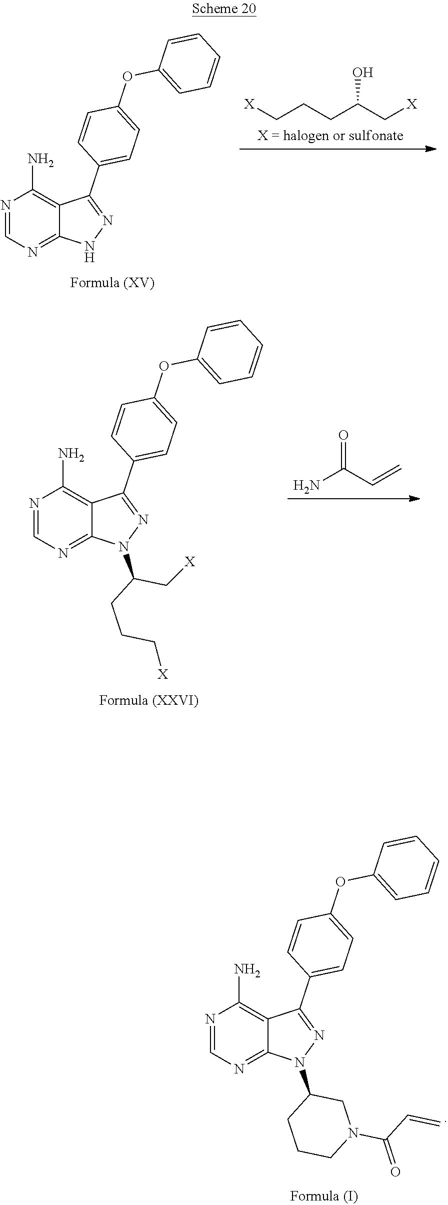

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
A) the coupling of the compound with the structure of Formula (XV),

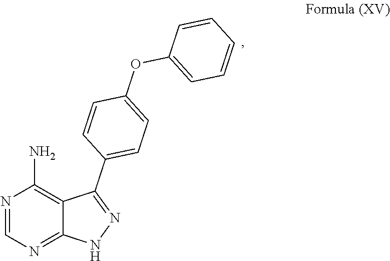 [SMILES: Nc1ncnc2[nH]nc(-c3ccc(Oc4ccccc4)cc3)c12]

with a compound with the structure

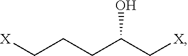

wherein X is halogen or sulfonate, to produce a compound with the structure of Formula (XXVI),

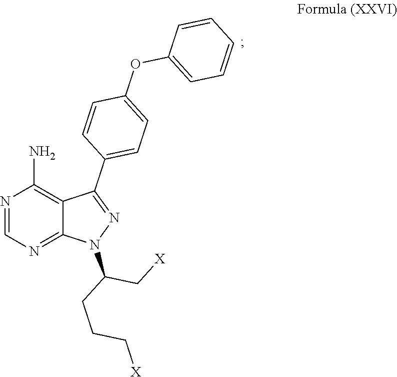

B) followed by the reaction of the compound with the structure of Formula (XXVI),

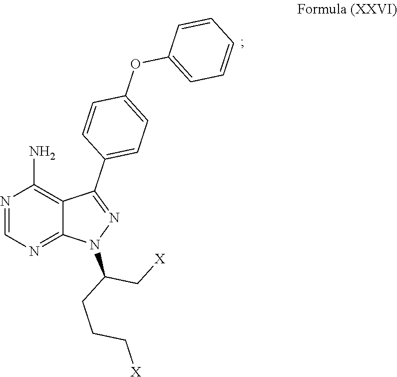

with acrylamide to produce a compound with the structure of Formula (I),

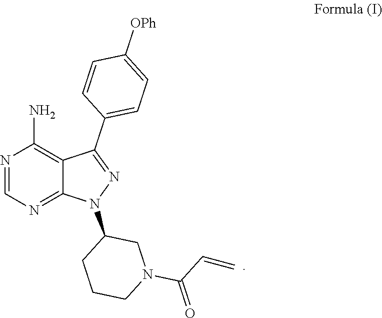 [SMILES: C=CC(=O)N1CCC[C@@H](n2nc(-c3ccc(Oc4ccccc4)cc3)c3c(N)ncnc32)C1]

In some embodiments of the process of Scheme 20, X is Cl. In some embodiments of the process of Scheme 20, X is Br. In some embodiments of the process of Scheme 20, X is trifluoromethanesulfonate. In some embodiments of the process of Scheme 20, X is methanesulfonate.

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), is outlined in Scheme 21:

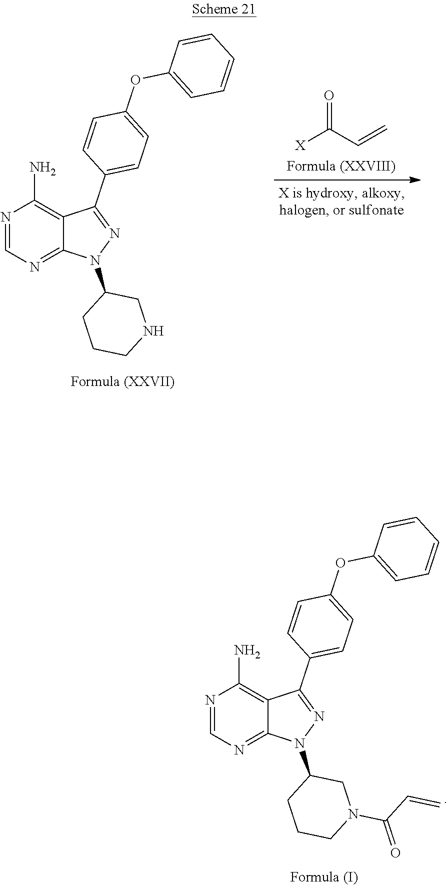

In some embodiments, described herein, the process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprises:
the coupling of a compound with the structure of Formula (XXVII),

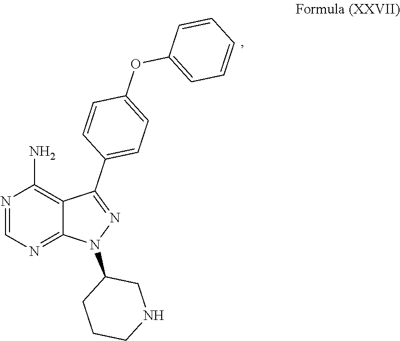 [SMILES: Nc1ncnc2c1c(-c1ccc(Oc3ccccc3)cc1)nn2[C@@H]1CCCNC1]

with a compound with the structure of Formula (XXVIII),

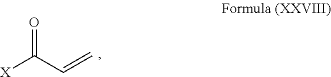

wherein X is a leaving group such as hydroxy, alkoxy, Br, sulfonate or dialkoxy-phosphoryl (P(═O)(OR⁴)₂ (each R⁴ is independently alkyl, e.g., Me or Et)), to produce a compound with the structure of Formula (I),

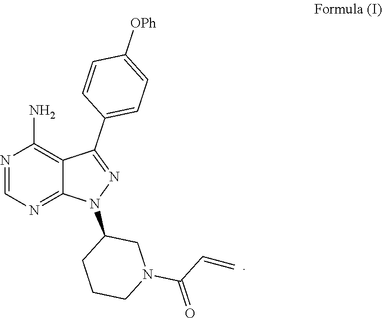 [SMILES: C=CC(=O)N1CCC[C@@H](n2nc(-c3ccc(Oc4ccccc4)cc3)c3c(N)ncnc32)C1]

In some embodiments of the process of Scheme 21, X is hydroxy. In some embodiments of the process of Scheme 21, X is alkoxy. In some embodiments of the process of Scheme 21, X is Br. In some embodiments of the process of Scheme 21, X is trifluoromethanesulfonate. In some embodiments of the process of Scheme 21, X is methanesulfonate. In some embodiments of the process of Scheme 21, X is P(═O)(OR⁴)₂, such as P(═O)(OMe)₂ or P(═O)(OEt)₂.

In general, the processes described herein, may have the advantage that the compounds prepared may be produced in a manner that utilizes fewer reagents and/or solvents, and/or requires fewer reaction steps (e.g. distinct/separate reaction steps) compared to processes disclosed in the prior art.

The process of the invention may also have the advantage that the compound(s) prepared is/are produced in higher yield, in higher purity, in higher selectivity (e.g. higher regioselectivity), in less time, in a more convenient (i.e. easy to handle) form, from more convenient (i.e. easy to handle) precursors, at a lower cost and/or with less usage and/or wastage of materials (including reagents and solvents) compared to the procedures disclosed in the prior art. Furthermore, there may be several environmental benefits of the process of the invention.

### Use of Protecting Groups

In the reactions described, it may be necessary to protect reactive functional groups, for example hydroxy, amino, imino, thio or carboxy groups, where these are desired in the final product, to avoid their unwanted participation in the reactions. Protecting groups are used to block some or all reactive moieties and prevent such groups from participating in chemical reactions until the protective group is removed. In one embodiment, each protective group may be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, or they may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

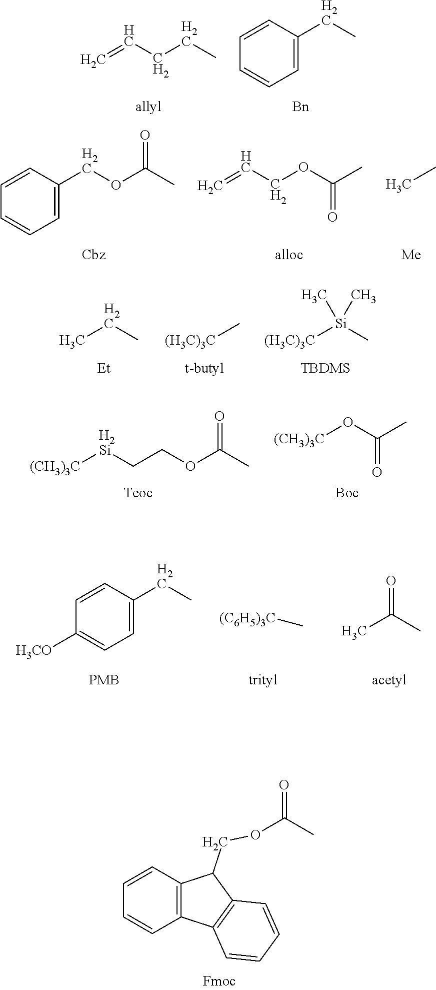

Amino protecting groups include, but are not limited to, mesitylenesulfonyl (Mts), benzyloxycarbonyl (Cbz or Z), 2-chlorobenzyloxycarbonyl, t-butyloxycarbonyl (Boc), t-butyldimethylsilyl (TBS or TBDMS), 9-fluorenylmethyloxycarbonyl (Fmoc), tosyl, benzenesulfonyl, 2-pyridyl sulfonyl, succinimide, pthalimide, p-methoxybenzyl (PMB), or suitable photolabile protecting groups such as 6-nitroveratryloxy carbonyl (Nvoc), 5-bromo-7-nitroindolinyl, nitrobenzyl, α-,α-dimethyldimethoxybenzyloxycarbonyl (DDZ), nitropiperonyl, pyrenylmethoxycarbonyl, and the like Amino protecting groups susceptible to acid-mediated removal include but are not limited to Boc and TBDMS Amino protecting groups resistant to acid-mediated removal and susceptible to hydrogen-mediated removal include but are not limited to allyloxycarbonyl, Cbz, nitro, and 2-chlorobenzyloxycarbonyl Amino protecting groups resistant to acid-mediated removal and susceptible base-mediated removal include but are not limited to Fmoc, (1,1-dioxobenzo[b]thiophene-2-yl)methyloxycarbonyl (Bsmoc), 2,7-di-tert-butyl-Fmoc, 2-fluoro-Fmoc (Fmoc(2F)), 2-(4-nitrophenylsulfonyl)ethoxycarbonyl (Nsc), (1,1-dioxonaphtho[1,2-b]thiophene-2-yl)methyloxycarbonyl (a-Nsmoc), 1-(4,4-dimethyl-2,6-dioxocyclohex-1-ylidene)-3-methylbutyl (ivDde), ethanesulfonylethoxycarbonyl (Esc), and 2-[phenyl(methyl)sulfonio]ethyloxycarbonyl tetrafluoroborate (Pms), tetrachlorophthaloyl (TCP), etc. Hydroxyl protecting groups include, but are not limited to, Fmoc, TBS, photolabile protecting groups (such as nitroveratryl oxymethyl ether (Nvom)), Mem (methoxyethoxy methyl ether), Mom (methoxy methyl ether), NPEOC (4-nitrophenethyloxycarbonyl) and NPEOM (4-nitrophenethyloxymethyloxycarbonyl).

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protecting Groups, Thieme Verlag, New York, N.Y., 1994, which are incorporated herein by reference in their entirety.

### Compound of Formula (I), and Pharmaceutically Acceptable Salts or Compositions Thereof

The Btk inhibitor compound described herein (i.e. compound of Formula (I)) is selective for Btk and kinases having a cysteine residue in an amino acid sequence position of the tyrosine kinase that is homologous to the amino acid sequence position of cysteine 481 in Btk. The Btk inhibitor compound can form a covalent bond with Cys 481 of Btk (e.g., via a Michael reaction).

A wide variety of pharmaceutically acceptable salts is formed from the compound of Formula (I) and includes:
acid addition salts formed by reacting the compound of Formula (I) with an organic acid, which includes aliphatic mono- and di-carboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;
acid addition salts formed by reacting the compound of Formula (I) with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to the compound of Formula (I) refers to a salt of the compound of Formula (I), which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but not limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of the compound of Formula (I), or pharmaceutically acceptable salts thereof, are conveniently prepared or formed during the processes described herein. In some embodiments, solvates of the compound of Formula (I) are anhydrous. In some embodiments, the compound of Formula (I), or pharmaceutically acceptable salts thereof, exist in unsolvated form. In some embodiments, the compound of Formula (I), or pharmaceutically acceptable salts thereof, exist in unsolvated form and are anhydrous.

In yet other embodiments, the compound of Formula (I), or a pharmaceutically acceptable salt thereof, is prepared in various forms, including but not limited to, amorphous phase, crystalline forms, milled forms and nano-particulate forms. In some embodiments, the compound of Formula (I), or a pharmaceutically acceptable salt thereof, is amorphous. In some embodiments, the compound of Formula (I), or a pharmaceutically acceptable salt thereof, is amorphous and anhydrous. In some embodiments, the compound of Formula (I), or a pharmaceutically acceptable salt thereof, is amorphous. In some embodiments, the compound of Formula (I), or a pharmaceutically acceptable salt thereof, is amorphous and anhydrous.

There is then further provided a process for the preparation of a pharmaceutical composition comprising ibrutinib, which process comprises bringing into association ibrutinib (or a pharmaceutically acceptable salt thereof), which is prepared in accordance with the processes described herein, with (a) pharmaceutically acceptable excipient(s), adjuvant(s), diluents(s) and/or carrier(s).

### Suitable Solvents

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethylene glycol, formamide, hexane, methanol, 2-methoxyethanol, methyl butyl ketone, methylcyclohexane, N-methylpyrrolidine, nitromethane, pyridine, sulfolane, tetralin, toluene, 1,1,2-trichloroethene, tetrahydrofuran and xylene.

Class 3 solvents, which possess low toxicity, include: acetic acid, acetone, anisole, 1-butanol, 2-butanol, butyl acetate, tert-butyl methyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methyl ethyl ketone, methyl isobutyl ketone, 2-methyl-1-propanol, pentane, 1-pentanol, 1-propanol, 2-propanol, and propyl acetate.

Residual solvents in active pharmaceutical ingredients (APIs) originate from the manufacture of API. In some cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising the compound of Formula (I) comprise an organic solvent(s). In some embodiments, compositions comprising the compound of Formula (I) comprise a residual amount of an organic solvent(s). In some embodiments, compositions comprising the compound of Formula (I) comprise a residual amount of a Class 3 solvent. In some embodiments, the organic solvent is a Class 3 solvent. In some embodiments, the Class 3 solvent is selected from the group consisting of acetic acid, acetone, anisole, 1-butanol, 2-butanol, butyl acetate, tert-butyl methyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methyl ethyl ketone, methyl isobutyl ketone, 2-methyl-1-propanol, pentane, 1-pentanol, 1-propanol, 2-propanol, and propyl acetate. In some embodiments, the Class 3 solvent is selected from ethyl acetate, isopropyl acetate, tert-butyl methyl ether, heptane, isopropanol, and ethanol.

### EXAMPLES

The following examples are intended to illustrate the present invention and should not be construed as a limitation of the scope of the present invention.

### Example 1. Compound XVII-A to Compound XVII-1 and Isolation of Compound XVII-1

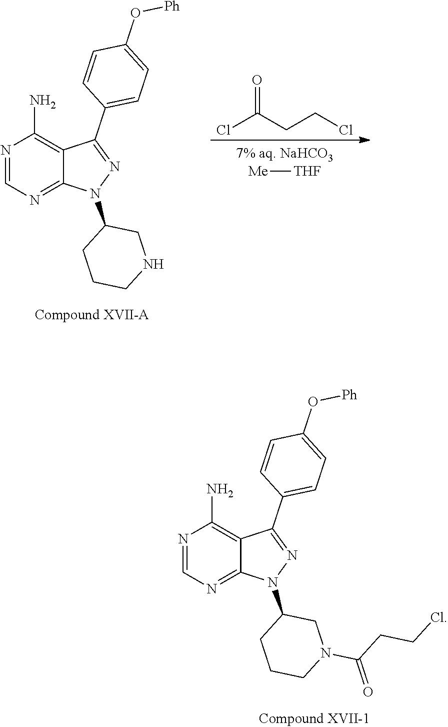

Compound XVII-A (80 g, 0.207 mol), 0.16 g of BHT (=butylated hydroxy toluene) and Me-THF (656.0 g) were added into a 2L jacket reactor equipped with over-head stirring. After stirring for 20 min at 10° C., 7% aq. NaHCO₃ (752 g, 0.627 mol) was added and then 3-chloropropionyl chloride (25.2 g, 0.198 mol) was slowly added via a dropping funnel over 1 h under nitrogen atmosphere/protection at 10° C. After stirring the reaction mixture at 10° C. for 1 h, the other part of 3-chloropropionyl chloride (2.61 g, 20.5 mmol) was diluted with Me-THF (32 g, 0.4×) and then slowly added into the reactor over 30 min at 10° C. After stirring for 30 min at 10° C., the aqueous phase was separated out and the Me-THF solution containing Compound XVII-1 was washed with 7% NaHCO₃ (200 g, 0.167 mol). Finally, 676.7 g 2-Me-THF solution of Compound XVII-1 (this is referred to below as Solution A) was obtained with a purity of 97.68 %.

There were two methods to isolate Compound XVII-1 as a solid: crystallization from Me-THF/n-heptane and crystallization from EtOAc/n-heptane. The detailed descriptions of crystallization of Compound XVII-1 from Me-THF/n-heptane and EtOAc/n-heptane are summarized below.

Crystallization from Me-THF/n-Heptane:

The Me-THF solution of Compound XVII-1 (obtained from 20 g of Compound XVII-A, HPLC purity: 97.68%; i.e. one quarter of Solution A referred to above) was added into a 500 mL jacket flask with mechanical stirring for azeotropic distillation. First, the Me-THF solution was concentrated to 4-5V under vacuum (jacket temperature: 28° C.) and then fresh and dried Me-THF (200 mL) was added. This distillation cycle was repeated two times and then distillation endpoint was 4-5V. The anti-solvent n-heptane (80 ml) was then slowly added into reactor over 2 h at 15° C. After being stirred for another 1-2 h at 15° C., the mixture was filtered and the cake was washed with 1V Me-THF/n-heptane (20 mL, v/v=1/1). After drying the wet cake at 35° C. for 16 hrs under vacuum, 23.25 g Compound XVII-1 was isolated as white solid with the HPLC purity of 98.36% in isolated yield of 88.7%.

Crystallization from EtOAc/n-Heptane:

The Me-THF solution of Compound XVII-1 (obtained from 20 g Compound XVII-A, HPLC purity: 97.68%; i.e. one quarter of Solution A referred to above) was added into a 500 mL jacket flask with mechanical stirring, and then it was concentrated to 4-5V under vacuum (jacket temperature: 28° C.). EtOAc (200 mL) was added into the residue and then the mixture was concentrated to 4-5V again. This distillation cycle was repeated three times and then a lot of white solid precipitated out. The anti-solvent n-heptane (80 ml) was then slowly added into reactor over 2 h at 15° C. After being stirred for another 1-2 h at 15° C., the mixture was filtered and the cake was washed with EA/n-heptane (20 mL, v/v=4/4). After drying the wet cake at 35° C. for 16 hrs under vacuum, 21.7 g Compound XVII-1 was isolated as white solid with the HPLC purity of 98.57% in isolated yield of 87.9%.

Characterizing Data for Compound XVII-1

Data may be obtained to characterize Compound XVII-1, for example mass spectrometry data, melting point and/or NMR (nuclear magnetic resonance) data (e.g. proton). In this case, case was obtained to characterize Compound XVII-1 by, amongst other things, NMR, which characterizing data is referred to in the Figures as follows:

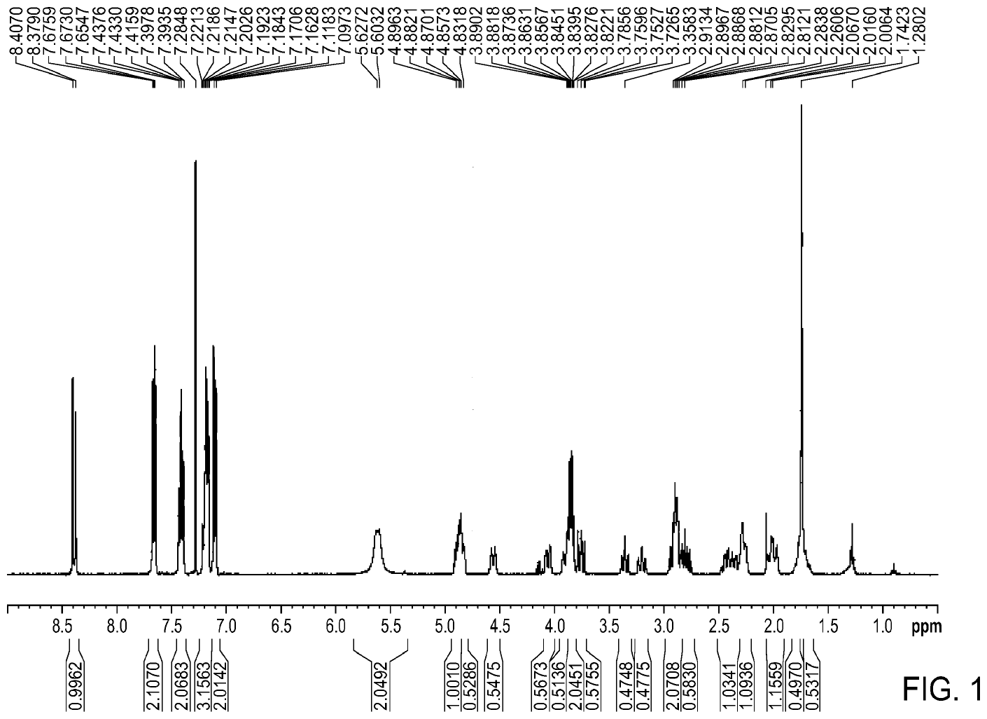

FIG. 1—¹H NMR of Compound XVII-1

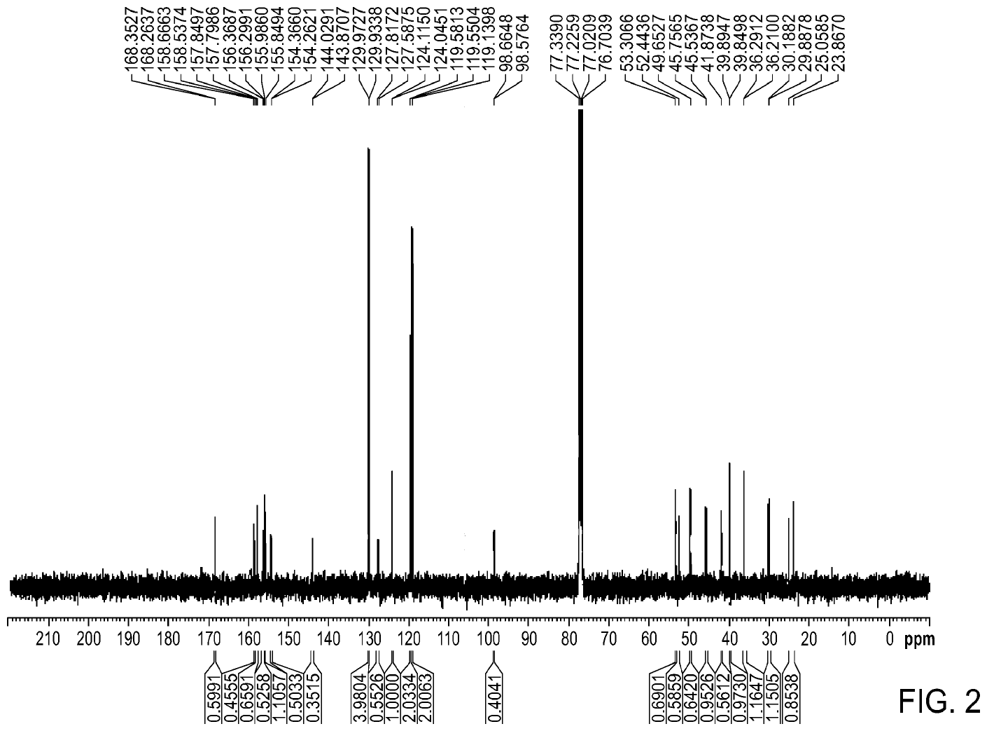

FIG. 2—¹³C NMR of Compound XVII-1

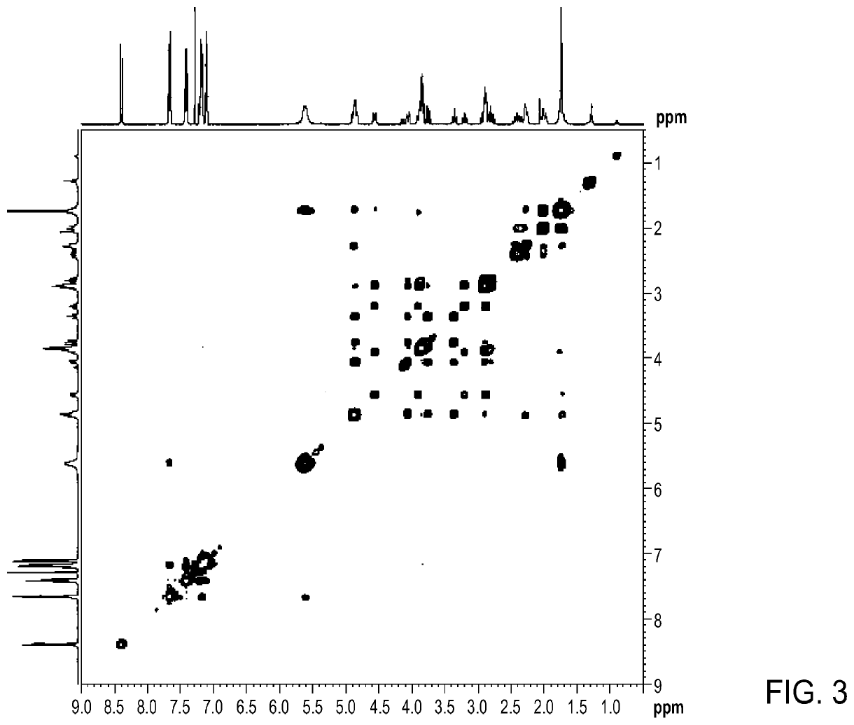

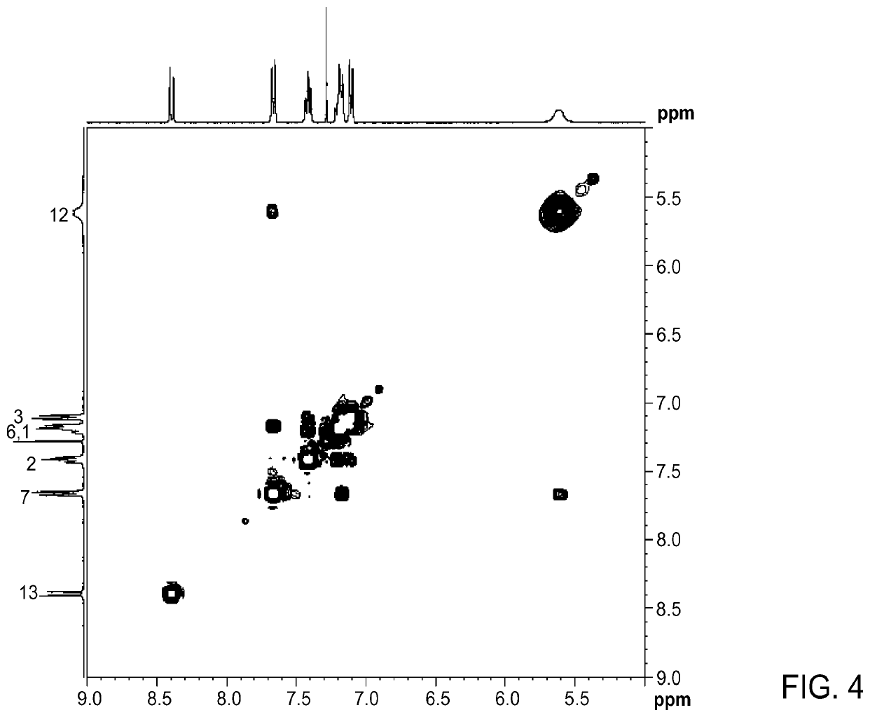

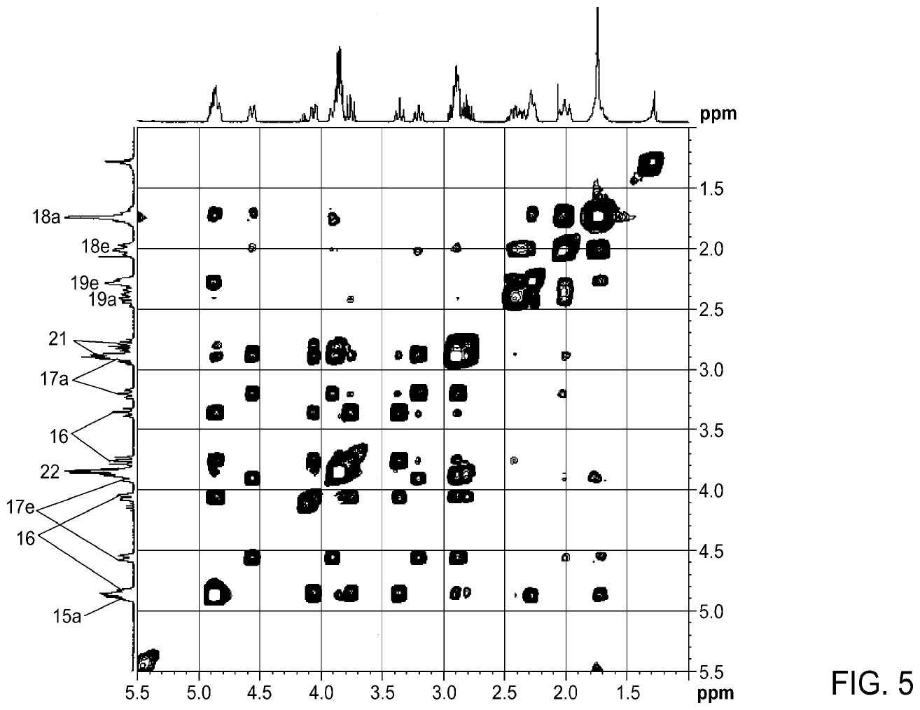

FIGS. 3, 4 and 5—NMR NOE (Nuclear Overhauser Effect) of Compound XVII-1

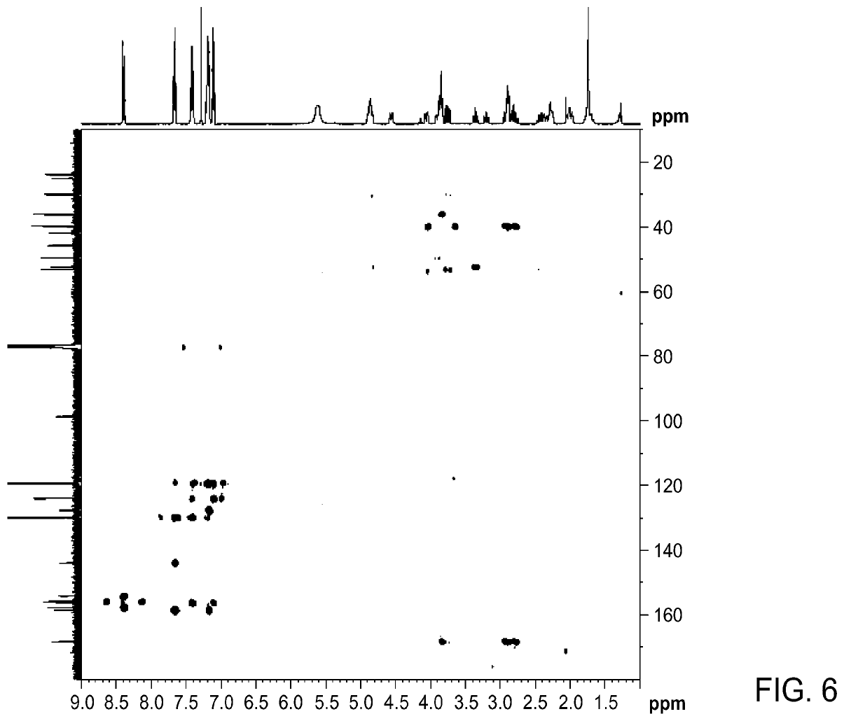

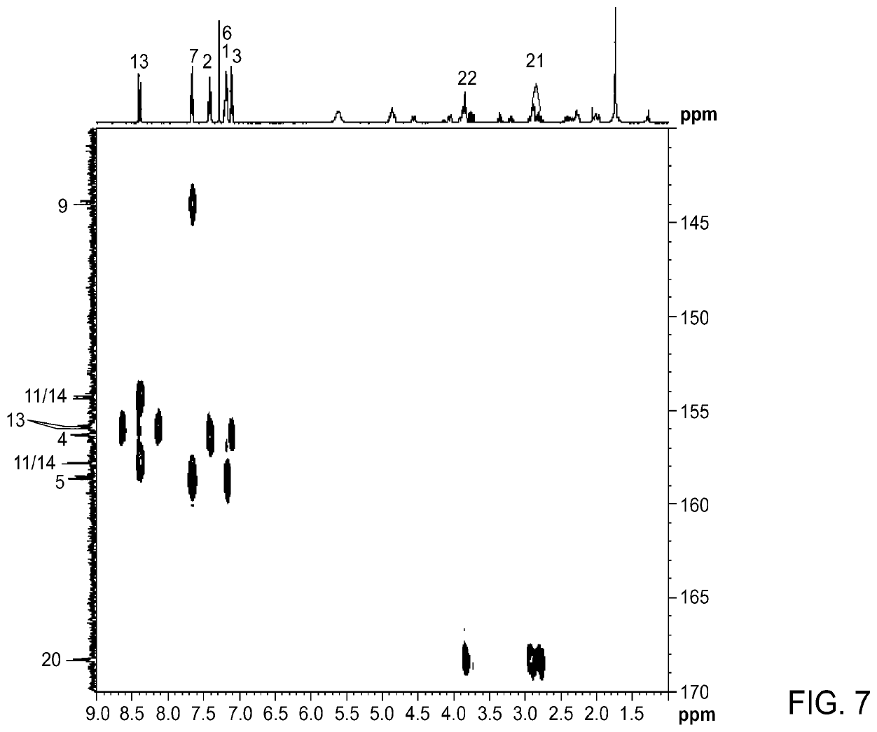

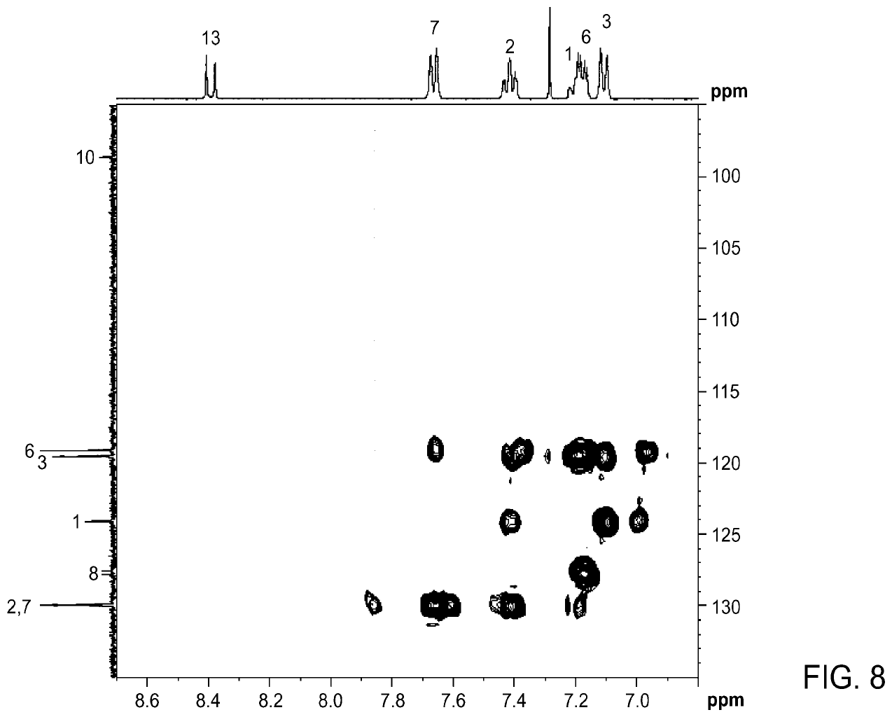

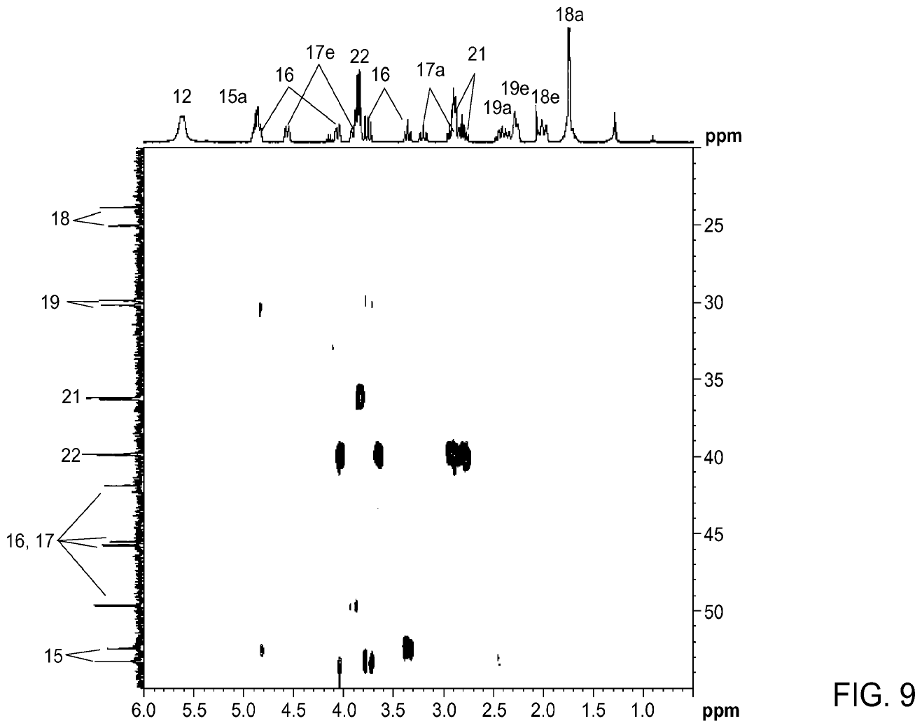

FIGS. 6, 7, 8 and 9—NMR HMBC (Heteronuclear Multiple-bond Correlation Spectroscopy) of Compound XVII-1

Where a NOE NMR is referred to, this is a spectroscopic method known to those skilled in the art. It is a two-dimensional NMR spectroscopy method. The NOE occurs through space (hence those atoms in close proximity will display a NOE) rather than the usual spin-spin coupling effects seen by proton and carbon NMR. Where a HMBC NMR is referred to, this is a specific spectroscopic method also known by those skilled in the art. It is also a two-dimensional NMR spectroscopy method. It is used to detect heteronuclear correlations over longer ranges of about 2-4 bonds.

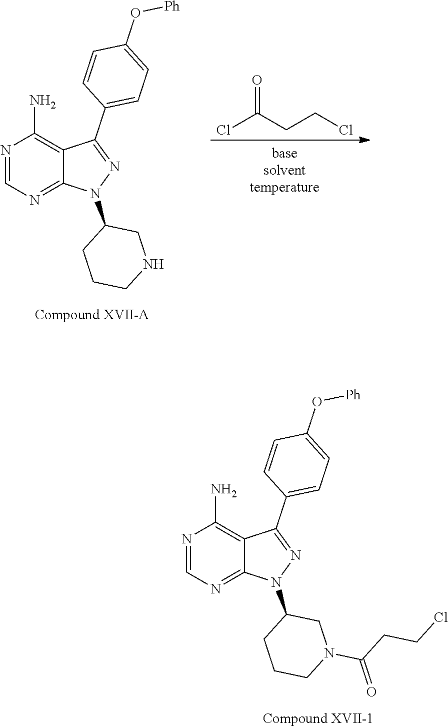 [SMILES: Nc1ncnc2c1c(-c1ccc(Oc3ccccc3)cc1)nn2[C@@H]1CCCNC1 ; O=C(Cl)CCCl ; Nc1ncnc2c1c(-c1ccc(Oc3ccccc3)cc1)nn2[C@@H]1CCCN(C(=O)CCCl)C1]

A screening exercise was done testing a variety of bases in this process reaction, and where the end-products as a result of the reaction were measured i.e. percentage of remaining starting material (Compound XVII-A), desired product (Compound XVII-1) and Compound I (i.e. ibrutinib) as a by-product.

Use of organic bases (3-CPC refers to 3-chloropropionyl chloride):

| Sol (V) | Base 5eq. | 3-CPC | Temp | Time | Cpd XVII-A (%) | Cpd XVII-1 (%) | Cpd I (%) |
|---|---|---|---|---|---|---|---|
| MeTHF | NMM | 1.06eq | | | 1.42 | 85.35 | 11.53 |
| 10 V | Lutidine | 1.12eq | 10° C. | 1 h | 0.78 | 92.01 | 5.75 |
| | Pyridine | 1.09eq | | | 27.47 | 68.02 | 0.85 |
| | NMM | 1.05eq | | | 11.59 | 76.43 | 10.32 |
| | Lutidine | 1.05eq | 40° C. | 1 h | 7.58 | 80.96 | 9.25 |
| | Pyridine | 1.05eq | | | 3.28 | 93.31 | 1.58 |

Use of inorganic bases: Schotten-Baumann conditions

| Sol (V) | Base 5eq. | 3-CPC | Temp | Time | Cpd XVII-A (%) | Cpd XVII-1 (%) | Cpd I (%) |
|---|---|---|---|---|---|---|---|
| MeTHF | Na₂CO₃ aq | 1.05eq. | 10° C. | 30 min | <0.05 | 94.0 | 4.7 |
| | NaHCO₃ aq | 1.05eq. | 10° C. | 30 min | 1.9 | 96.8 | 1.2 |
| EtOAc | Na₂CO₃ aq | 1.05eq. | 20° C. | 30 min | 2.7 | 92.2 | 5.0 |
| | NaHCO₃ aq | 1.05eq. | 10° C. | 30 min | 3.0 | 95.0 | 1.7 |

### Example 2 Compound XVII-1 to Compound I (Ibrutinib) and “One-Step” Method of Compound XVII-A to Compound I

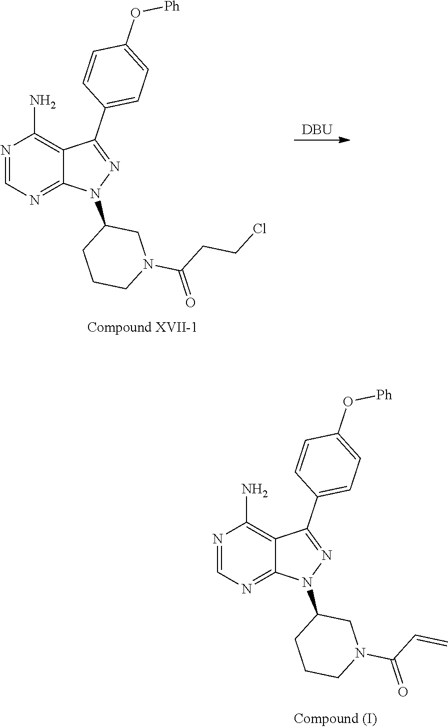 [SMILES: Nc1ncnc2c1c(-c1ccc(Oc3ccccc3)cc1)nn2[C@@H]1CCCN(C(=O)CCCl)C1 ; "DBU" ; C=CC(=O)N1CCC[C@@H](n2nc(-c3ccc(Oc4ccccc4)cc3)c3c(N)ncnc32)C1]

A 24.7 g batch of Compound XVII-1 was employed for the preparation of crude Compound I (ibrutinib). Firstly, Compound XVII-1 (in solid form) was added into 12V anhydrous EA (ethyl acetate), and then 2.5 eq DBU was added over 1 h at 20° C. After stirring at 20° C. for 24 hrs, the solution yielded 89% of the desired product.

Isolated Compound XVII-1 to Compound I, Using CF₃COONa

Procedure:
Charge 10 g Compound XVII-1 into R1 (reaction vessel 1)
Charge 115 ml EA (ethyl acetate) into R1
Charge 1.0 eq CF₃COONa into R1 and then add drop wise 2.5 eq DBU into R1 at 15° C. over 1 hr.
Rinse drop funnel with 5 ml EA
Stir R1 for 5 hrs at 15° C., and take a HPLC reading
Add drop-wise 11× (2.0 eq) 5% Na₂CO₃ into R1 within 0.5 h and then stir R1 for 1 h and then separate the phases
Wash the organic phase with 4.5× H₂O, maintain R₁ at 20° C. for 14 hr.
Separate the phases
Wash the organic with 3.0× 22% citric acid three times
Combine the aqueous phases and then extract it with 7V EA
Combine the organic layers
Wash the organic phase with 4.0× 10% Na₂CO₃ (pH=6.10) and then wash the organic phase with 4.5× H₂O twice
Obtain 143.36 g organic phase
After final workup and crystallization, 9.21 g crude Compound I was isolated in yield of 80.8%.
   From Compound XVII-A to Compound I, without Isolation of Compound XVII-1, with Elimination in Me-THF

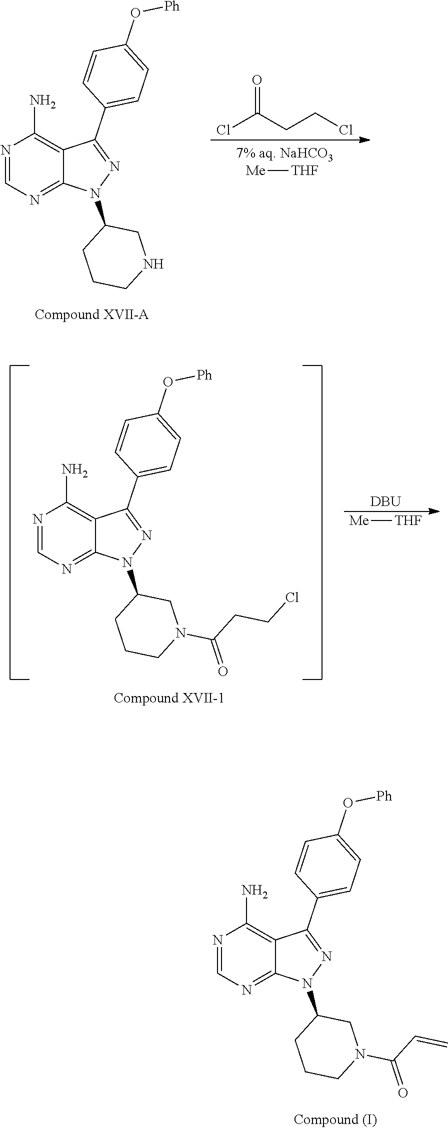

Procedure:
1. Charge Compound XVII-1 solution of Me-THF into R1 (20 g size based on Compound I; one quarter of Solution A as referred to above) without isolating Compound XVII-1
2. Concentrate the solution to 5.5V and then charge 4.5V 2-Me-THF to R1
3. Concentrate the solution to 5.5V and then charge 4.5V 2-Me-THF to R1
4. Concentrate the solution to 5.5V and then charge 4.5V 2-Me-THF to R1
5. Concentrate the solution to 5.5V and then charge 4.5V 2-Me-THF to R1
6. Concentrate the solution to 5.5V and then charge 6.5V 2-Me-THF to R1
7. Add drop wise 2.5 eq DBU into R1 at 22° C. for 1 hr
8. Stir R1 for 22 hrs at 22° C., transfer the mixture in R1 to R2
9. Wash the R1 with 1V 2-Me-THF and then transfer to R2
10. Wash the organic phase(s) with 3.0× 22% citric acid and then separate the phases. Wash the organic with 3.0× 22% citric acid and then separate the phases
11. Wash the organic with 3.0× 22% citric acid and then separate the phases. Combine the aqueous phases and then extract it with 7V 2-Me-THF. The HPLC purity of the organic phase(s) is measured
12. Combine the aqueous phases and obtain 161.24 g aqueous phases
13. Combine the organic layers
14. Wash the organic phase with 8.4× 10% Na₂CO₃ (pH=6-7.5)
15. Wash the organic phase with 4.5× H₂O twice
16. Obtain 343.23 g organic phase
17. After final workup and crystallization, 17.44 g crude Compound I was isolated in yield of 76.5%

From Compound XVII-A to Compound I, without Isolation of Compound XVII-1, in EA, without addition of CF₃COONa

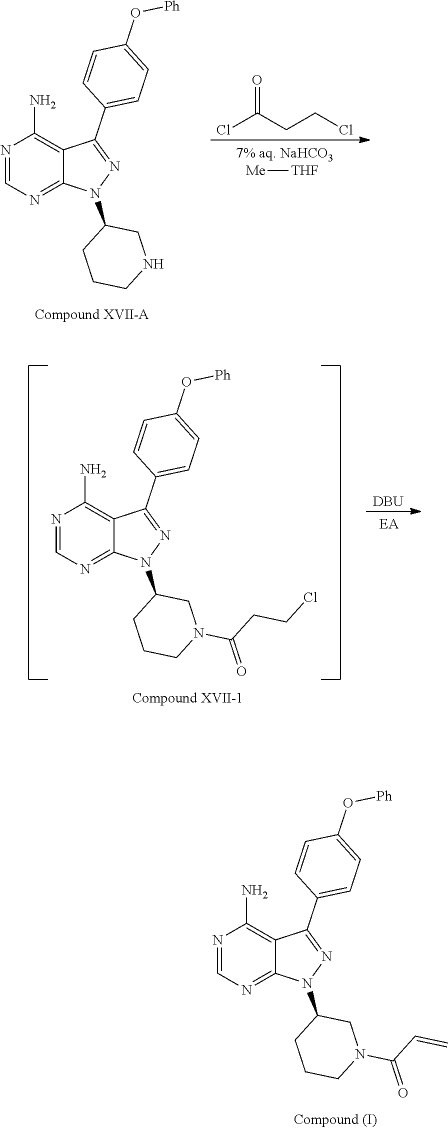

Procedure:

Charge Compound XVII-1 solution of Me-THF into R1 (20 g size based on Compound I; one quarter of Solution A as referred to above) without isolating Compound XVII-1
Concentrate the solution to 5.5V and then charge 4.5V EA to R1
Concentrate the solution to 5.5V and then charge 4.5V EA to R1
Concentrate the solution to 5.5V and then charge 4.5V EA to R1
Concentrate the solution to 5.5V and then charge 4.5V EA to R1
Concentrate the solution to 5.5V and then charge 6.5V EA to R1
Add drop wise 2.5 eq DBU into R1 at 22° C. over 1 hr
Stir R1 for 22 hrs at 22° C., transfer the mixture in R1 to R2
Wash the R1 with 1V EA and then transfer to R2
Wash the organic with 3.0× 22% citric acid and then separate the phases. Wash the organic with 3.0× 22% citric acid and then separate the phases
Wash the organic with 3.0× 22% citric acid and then separate the phases. Combine the aqueous phases and then extract it with 7V EA
Combine the aqueous phases and obtain 190.59 g aqueous phases
Combine the organic layers
Wash the organic phase with 3.8× 10% Na₂CO₃ (pH=6-7.5)
Wash the organic phase with 4.5× H₂O twice
Obtain 360.48 g organic phase
After final workup and crystallization, 16.70 g crude Compound I was isolated in yield of 73.2% (yield loss in mother liquor was 6.3%)

From Compound XVII-A to Compound I, without Isolation of Compound XVII-1, with Addition of CF₃COONa

Procedure:
Compound XVII-1 solution of Me-THF into R1 (20 g size based on Compound I; one quarter of Solution A as referred to above) without isolating Compound XVII-1
Concentrate the solution to 5.5V and then charge 4.5V EA to R1
Concentrate the solution to 5.5V and then charge 4.5V EA to R1
Concentrate the solution to 5.5V and then charge 4.5V EA to R1
Concentrate the solution to 5.5V and then charge 4.5V EA to R1
Concentrate the solution to 5.5V and then charge 6.5.5V EA to R1
Charge 1.0 eq CF₃COONa (7.2 g) into R1
Add drop wise 2.5 eq DBU (19.6 g) into R1 at 15° C. over 1 hr
Stir R1 for 3 hrs at 15° C., and transfer the mixture in R1 to R2
Stir the mixture in R2 for 3 h
Add drop-wise 2 eq 5% Na₂CO₃ into R1 over 0.5 h
Stir R1 for 1 h
Separate the mixture solution in R1
Wash the organic phase with 4.5× H₂O
Wash the organic with 3.0× 22% citric acid three times, W=197 g, assay is 0.32%, loss yield is 2.76%.
Combine the aqueous phase(s) and extract it with 7V EA
Combine the organic phase(s) and adjust pH to 6-7.5 with 10% Na₂CO₃ (3.9×)
Wash the organic phase(s) with 4.5× H₂O twice. Solution yield was 91.64%

### Screening of Additives in the Elimination Step

Compound XVII-1 (12V; ethyl acetate)→1.0 eq. additive→stir 10-15 min→dropwise addition of 2.5 eq. DBU over 1 hr→stir at 22° C. (reaction time)→Compound I

| | | | residual | Compound I in | |
|---|---|---|---|---|---|
| | | | Compound | the solution | |
| | Aspect of reaction | Reaction | XVII-1 | after work-up | |
| Additive | mixture | time (h) | HPLC area % | HPLC area % | Obs |
| none | Sticky suspension | 22 | 0.63 | 98.24 | |
| CF₃COONa | Light, easy stirrable | 3 | 0 | 99.77 | Solution yield |
| | suspension | | | | 91.64% |
| CH₃COONa | Stirrable suspension | 17 | 0.02 | 99.42 | Isolated yield |
| | | | | | 83.2 |
| CH₃COONa•3H₂O | Light suspension | 4 | 0.016 | 99.06 | |
| Na lactate | Heavy suspension | 26 | nd | 99.60 | |
| CH₃SO₃Na | Heavy suspension | 26 | 0.48 | 99.11 | |
| CF₃SO₃Na | Light, somewhat | 6 | nd | 76.07 | |
| | sticky suspension | | | | |
| CF₃SO₃Li | Suspension, | 20 | 0.54 | 73.03 | |
| | solid and oil | | | | |

### Screening of Bases and Conditions for Effecting the Elimination

Compound XVII-1→base, solvent, temperature, reaction time→Compound I

| | Solvent, | Reaction time | Residual Cpd XVII-1 | Compound I in the |
|---|---|---|---|---|
| Base (eqs) | Temperature (° C.) | (h) | HPLC area % | soln. HPLC area % |
| DBU (2) | EtOAc | 7 | 0.02 | 98.64 |
| | 30 | | | |
| DBU (4) | EtOAc | 22 | 0.62 | 92.41 |
| | 20 | | | |
| DBU (5) | EtOAc | 22 | 1.17 | 92.35 |
| | 20 | | | |
| Et₃N (5) | EtOAc | 22 | 87.32 | 11.65 |
| | 65 | | | |
| Et₃N (5) | EtOAc | 22 | 64.71 | 33.49 |
| | 65 | | | |
| NMM (5) | EtOAc | 7 | 98.09 | 1.33 |
| | 30 | | | |
| NMM (5) | EtOAc | 7 | 96.95 | 2.45 |
| KOtBu (2) | MeTHF | 6 | 0 | 3.02 |
| | 25 | | | |
| DMAP (1) | MeTHF | 6 | 0.049 | 96.85 |
| DBU (2) | 25 | | | |
| NaOH (2) | MeTHF | 20 | 0 | 15.28 |
| DBU (1) | 25 | | | |
| DABCO (1) | MeTHF | 20 | 0 | 96.19 |
| DBU (2) | 25 | | | |
| LiHMDS (2) | MeTHF | 3 | 0 | 48.76 |
| | 0 | | | |

### Example—Pharmaceutical Formulation

Ibrutinib may be formulated into a pharmaceutically acceptable formulation using standard procedures.

For example, there is provided a process for preparing a pharmaceutical formulation comprising ibrutinib, or a derivative thereof, which process is characterised in that it includes as a process step a process as hereinbefore defined. The skilled person will know what such pharmaceutical formulations will comprise/consist of (e.g. a mixture of active ingredient (i.e. ibrutinib or derivative thereof) and pharmaceutically acceptable excipient, adjuvant, diluent and/or carrier).

There is further provided a process for the preparation of a pharmaceutical formulation comprising ibrutinib (or a derivative thereof), which process comprises bringing into association ibrutinib, or a pharmaceutically acceptable salt thereof (which may be formed by a process as hereinbefore described), with (a) pharmaceutically acceptable excipient(s), adjuvant(s), diluent(s) and/or carrier(s).

The examples and embodiments described herein are illustrative and various modifications or changes suggested to persons skilled in the art are to be included within this disclosure.

1-45. (canceled)

46. A process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising reacting a compound of Formula (XXVII) with a compound of Formula (XXVIII), wherein X is a leaving group selected from the group consisting of hydroxy, alkoxy, sulfonate, and P(═O)(OR⁴)₂, wherein each R⁴ is independently alkyl:

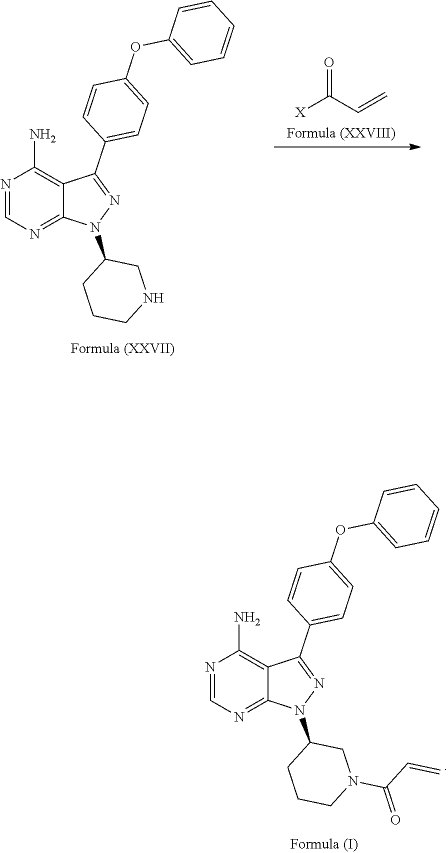

47-48. (canceled)

49. The process according to claim 46, wherein X is hydroxy.

50. The process according to claim 46, wherein X is alkoxy.

51. The process according to claim 46, wherein X is trifluoromethanesulfonate or methanesulfonate.

52. The process according to claim 46, wherein X is P(═O)(OR⁴)₂.

53. The process according to claim 52, wherein X is P(═O)(OMe)₂ or P(═O)(OEt)₂.

54. A compound according to Formula (XVII): 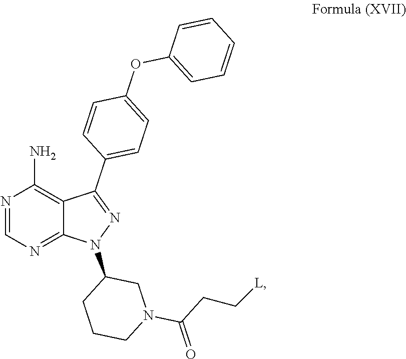
wherein L is selected from the group consisting of Br, I, hydroxy, alkoxy, sulfonate, phosphate, substituted phosphate or dialkoxyphosphoryl.

55. The compound according to claim 54, wherein L is Br, I, hydroxy, alkoxy, methanesulfonate, or trifluoromethanesulfonate.

56. The compound according to claim 54, wherein L is Br or I.

57. The compound according to claim 54, wherein L is hydroxy.

58. The compound according to claim 54, wherein L is alkoxy.

59. The compound according to claim 54, wherein L is trifluoromethanesulfonate.

60. A process for the preparation of 1-((R)-3-(4-amino-3-(4-phenoxyphenyl)-1H-pyrazolo[3,4-d]pyrimidin-1-yl)piperidin-1-yl)prop-2-en-1-one (ibrutinib), wherein ibrutinib is the compound of Formula (I), comprising a beta-elimination of a compound with the structure of Formula (XVII), wherein L is a leaving group selected from the group consisting of hydroxy, alkoxy, methanesulfonate, and trifluoromethanesulfonate: 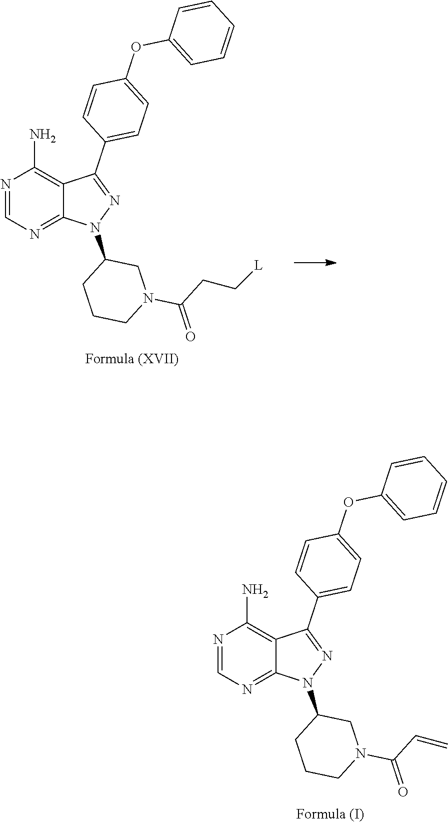
wherein the beta-elimination is carried out at a reaction temperature between about 0° C. and about 60° C. and in the presence of at least one equivalent of base, for a period between about 1 hour and about 24 hours.

61. The process according to claim 60, wherein the base is present at a ratio of at least 1.5 equivalents base.

62. The process according to claim 61, wherein the base is present at a ratio between about 2 equivalents and about 5 equivalents.

63. The process according to claim 60, wherein the base is an organic base or an inorganic base.

64. The process according to claim 63, wherein the organic base is selected from the group consisting of an alkoxide base, an amine base, an amide base, or a mixture thereof.

65. The process according to claim 64, wherein the amine base is 1,8-diazabicylco[5.4.0]undec-7-ene (DBU).

66. The process according to claim 60, wherein L is hydroxy.

67. The process according to claim 60, wherein L is alkoxy.

68. The process according to claim 60, wherein L is trifluoromethanesulfonate.

69. The process according to claim 60, wherein the compound with the structure of Formula (XVII) has an HPLC purity is greater than 50%.

70. The process according to claim 69, wherein the compound with the structure of Formula (XVII) has an HPLC purity is greater than 80%.

71. The process according to claim 69, wherein the compound with the structure of Formula (XVII) has an HPLC purity is greater than 90%.
